(19) United States
(12) Patent Application Publication
(10) Pub. No.: US 2008/0234301 A1
(43) Pub. Date: Sep. 25, 2008

(54) Alpha-4 Integrin Mediated Cell Adhesion Inhibitors for the Treatment or Prevention of Inflammatory Diseases

(76) Inventors: Robert William Ward, Essex (GB); Jason Witherington, Essex (GB)

Correspondence Address: BIRCH STEWART KOLASCH & BIRCH, PO BOX 747, FALLS CHURCH, VA, 22040-0747, US

(21) Appl. No.: 10/588,235

(22) PCT Filed: Feb. 8, 2005
(86) PCT No.: PCT/JP05/02194

§ 371 Date: Aug. 3, 2006

(30) Foreign Application Priority Data
Feb. 9, 2004 (GB) 0402812.2

(51) Int. Cl. A61K 31/513; C07D 239/36; A61K 31/50; C07D 237/14; A61P 9/08; A61P 35/00; A61P 37/04; A61P 1/02; A61P 37/02; A61P 3/10; A61P 25/28; A61P 9/10

(52) U.S. Cl. 514/269; 544/319; 544/239; 514/247

(57) ABSTRACT

The present invention relates to novel compounds of formula (I), processes for their preparation, compositions comprising them and their use in the treatment or prevention of diseases capable of being modulated by the inhibition of cell adhesion.

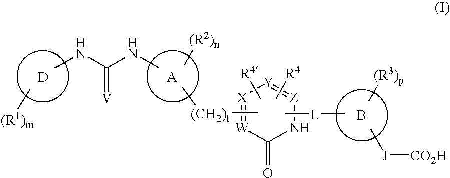

### TECHNICAL FIELD

The present invention relates to novel compounds, processes for their preparation, compositions comprising them and their use in the treatment or prevention of diseases capable of being modulated by the inhibition of cell adhesion. More particularly, the present invention relates to novel heterocyclic compounds that inhibit α₄ integrin mediated cell adhesion and which are believed to be useful for the treatment or prevention of inflammatory diseases.

### BACKGROUND ART

The multiple adhesive interactions between leukocytes and endothelial cells or extracellular matrix proteins are a key factor in the regulation of immunity and inflammation. The earliest events in the migration of leukocytes out of the vasculature at the site of inflammation include leukocyte rolling followed by changes in integrin avidity, which lead to subsequent firm adhesion (for reviews see Butcher, Cell 67:1033-1036 (1991); Harlan, Blood 3:513-525 (1985); Hemler, Annu. Rev. Immunol. 8:365-400 (1990); Osborn, Cell 62:3-6 (1990); Shimizu et al., Immunol. Rev. 114:109-143 (1990); Springer, Nature 346:425-434 (1990); and Springer, Cell 76:301-314 (1994)). In response to chemotactic factors, the leukocytes migrate through two adjacent endothelial cells and into tissues that are composed, in part, of the extracellular matrix protein fibronectin (FN) (see Wayner et al., J. Cell Biol. 105:1873-1884 (1987)) and collagen (CN) (see Bornstein et al., Ann. Rev. Biochem. 49:957-1003 (1980); and Miller, Chemistry of the collagens and their distribution, in “Extracellular Matrix Biochemistry”, K. A. Piez and A. H. Reddi, editors, Elsevier, Amsterdam, 41-78 (1983)). Important recognition molecules that participate in these adhesive reactions belong to the integrin gene superfamily (for reviews see Hemler, Annu. Rev. Immunol. 8:365-400 (1990); Hynes, Cell 48:549-554 (1987); Shimizu et al., Immunol. Rev. 114:109-143 (1990); and Springer, Nature 346:425-434 (1990)).

Integrins are heterodimers composed of non-covalently associated subunits, referred to as the alpha (α) and beta (β) subunits. To date, 8 integrin β subunits have been identified which can associate with 18 distinct α subunits to form 24 distinct integrins (see Hynes, Cell 110: 673-687 (2002)).

The α₄β₁ integrin, also known as VLA-4 (Very Late Antigen-4), is constitutively expressed on the surface of leukocytes including lymphocytes, monocytes, eosinophils and basophils (see Hemler et al., J. Bio. Chem. 262:11478-11485 (1987); and Bochner et al., J. Exp. Med. 173:1553-1556 (1991)). VLA-4 is reported to be present on neutrophils from septic patients (see Ibbotson et al., Nature Med. 7:465-470 (2001)). VLA-4 binds to vascular cell adhesion molecule-1 (VCAM-1) on activated endothelial cells, resulting in extravasation of leukocytes (Elices et al., Cell 60:577-584 (1990)). Once the cells have reached the extravascular space, VLA-4 can bind to the connecting segment 1 (CS-1), an alternatively spliced region of the FN A chain (Wayne et al., J. Cell Biol. 109:1321-1330 (1989)). In addition, VLA-4 is known to bind to osteopontin, a protein upregulated in arteriosclerotic plaques (see Bayless et al., J. Cell Science 111:1165-1174 (1998)).

Patent application PCT/JP03/10119 discloses a series of pyridone compounds that inhibit α₄ integrin mediated cell adhesion and which are useful for the treatment of chronic inflammatory diseases.

### DISCLOSURE OF INVENTION

A novel series of compounds has now been found which also inhibit α₄ integrin mediated cell adhesion. The present invention therefore provides, in a first aspect, a compound of formula (I) or a pharmaceutically acceptable derivative thereof:

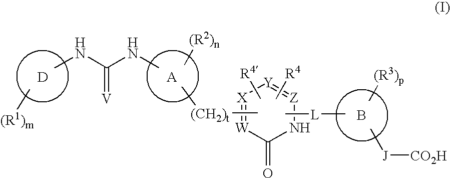

wherein
A, B and D are independently aryl or heteroaryl;
R¹, R² and R³ are independently C₁₋₆alkyl, halogen, C₁₋₆alkoxy, hydroxy, cyano, CF₃, OCF₃, nitro, C₁₋₆alkylthio, amino, mono- or di-C₁₋₆alkylamino, carboxy, C₁₋₆alkanoyl, amido, mono or di-C₁₋₆alkyl amido, —NHCOR⁹ or —NHSO₂R⁹ {in which R⁹ is C₁₋₆alkyl, C₃₋₇cycloalkyl or phenyl (optionally substituted by up to three groups selected from C₁₋₆alkyl, halogen, C₁₋₆alkoxy, cyano, phenyl and CF₃)} or is a group -E-(CH₂)₁₋₆NR^{x}R^{y} (in which E is a single bond or —OCH₂— and R^{x} and R^{y} are independently hydrogen, C₁₋₆alkyl or combine together to form a 5-7 membered heterocyclic ring); R⁴ and R^{4′} are independently hydrogen, C₁₋₆alkyl, halogen or C₁₋₆alkoxy;
V is O, S, NH, N—C₁₋₆alkyl, NNO₂ or NCN;
W, X, Y and Z are independently C, CH or N, subject to the proviso that at least one of X, Y and Z is N;
L is —(CH₂)_{q}— or —(CH₂)_{q}′O— where q is 0, 1, 2 or 3 and q′ is 2 or 3;

### J is

(i) a group —CR⁵═CR⁶— where R⁵ and R⁶ are independently hydrogen or C₁₋₆alkyl;
(ii) a group —CHR⁷—CHR⁸— where R⁷ and R⁸ are independently hydrogen, C₁₋₆alkyl, C₃₋₇cycloalkyl, aryl, heteroaryl, a group —NHCOR⁹ or —NHSO₂R⁹ in which R⁹ is as defined above or a group —(CH₂)₁₋₆NR^{x}R^{y} in which R^{x} and R^{y} are as defined above;
(iii) a single bond;
(iv) —CHR⁶— where R⁶ is as defined above;
(v) a group —O—CHR¹⁰—, —NR¹¹—CHR¹⁰— or —CR¹²R¹³—CHR¹⁰— where R¹⁰ and R¹¹ are independently hydrogen or C₁₋₆alkyl and R¹² and R¹³ are independently C₁₋₆alkyl or R¹² and R¹³ combine together to form a C₃₋₇cycloalkyl or a 5-7 membered heterocyclic ring;
   m, n and p are independently 0, 1, 2 or 3; and
   t is 0, 1 or 2.

In one embodiment the invention provides a compound of formula (I′) or a pharmaceutically acceptable derivative thereof:

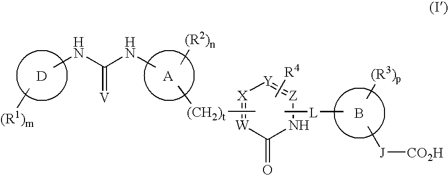

in which R¹-R⁴, m, n, p, t, A, B, D, L, J, V, W, X, Y and Z are as defined in formula (I).

A particularly preferred sub-class of the compound of formula (I) is a compound of formula (Ia) or a pharmaceutically acceptable derivative thereof:

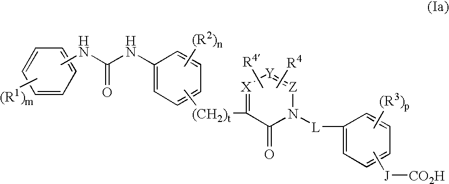

wherein:
R¹-R⁴, R^{4′}, L, J, X, Y, Z, m, n, p and t are as defined in formula (I).

In a further embodiment the invention provides a compound of formula (Ia′) or a pharmaceutically acceptable derivative thereof:

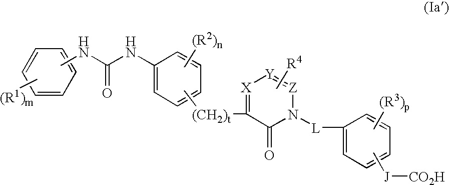

in which:
R¹-R⁴, L, J, X, Y, Z, m, n, p and t are as defined in formula (I).
Throughout the present specification, unless otherwise stated:
the term “halogen” is used to describe a group selected from fluorine, chlorine, bromine and iodine;
the term “C₁₋₆alkyl” is used to describe a group or a part of the group comprising a linear or branched alkyl group containing from 1 to 6 carbon atoms; examples of such groups include methyl, ethyl, propyl, isopropyl, n-butyl, isobutyl, tert-butyl, pentyl or hexyl;
the term “aryl” is used to denote phenyl and naphthyl (naphth-1-yl and naphth-2-yl) groups;
the term “heteroaryl” is intended to mean an aromatic or a benzofused aromatic ring containing 1 to 3 heteroatoms selected from oxygen, nitrogen and sulphur. Suitable examples of such aromatic rings include thienyl, furyl, pyrrolyl, triazolyl, imidazolyl, oxazolyl, thiazolyl, oxadiazolyl, isothiazolyl, isoxazolyl, thiadiazolyl, pyridonyl, pyrazolyl, pyrimidinyl, pyridazinyl, pyrazinyl and pyridyl. Suitable examples of such benzofused aromatic rings include quinolyl, isoquinolyl, indolyl, benzofuranyl, benzothiophenyl, benzimidazolyl and benzoxazolyl;
the term “5-7 membered heterocyclic ring” is intended to mean a non-aromatic heterocyclic ring comprising 1-3 heteroatoms selected from nitrogen, oxygen and sulphur. Suitable examples of such rings include piperidyl, piperazinyl, pyrrolidinyl and morpholinyl and the like. The heterocyclic rings are optionally substituted by C₁₋₆alkyl;
the term “C₁₋₆ alkoxy” is used to describe a group or a part of the group wherein an oxygen atom is bound to the above-mentioned alkyl group; examples of such groups include methoxy, ethoxy, propoxy, isopropoxy, butoxy, isobutoxy, sec-butoxy and tert-butoxy, pentoxy or hexoxy;
the term “C₁₋₆ alkanoyl” is used to describe groups formed by removing a “OH” group from the carboxyl group of a C₁₋₆ carboxylic acid; examples of such groups include formyl, acetyl, propionyl or butyryl;
the term “C₃₋₇ cycloalkyl” means a cyclic C₃₋₇ alkyl group; examples of such groups include cyclohexyl or cyclopentyl;

### BEST MODE FOR CARRYING OUT THE INVENTION

When A, B and/or D is aryl a preferred group is phenyl. When A, B and/or D is heteroaryl a preferred group is pyridyl.

Suitably, A is phenyl or pyridyl.

Suitably, B is phenyl.

Suitably D is phenyl or pyridyl.

Suitably, R¹, R² and R³ are independently C₁₋₆alkyl, halogen, C₁₋₆alkoxy, hydroxy, cyano, CF₃, nitro, C₁₋₆alkylthio, amino, mono- or di-C₁₋₆alkylamino, carboxy, C₁₋₆alkanoyl, amido, mono or di-C₁₋₆alkyl amido, —NHCOR⁹ or —NHSO₂R⁹ {in which R⁹ is C₁₋₆alkyl, C₃₋₇cycloalkyl or phenyl (optionally substituted by up to three groups selected from C₁₋₆alkyl, halogen, C₁₋₆alkoxy, cyano, phenyl or CF₃)} or is a group -E-(CH₂)₁₋₆NR^{x}R^{y} (in which E is a single bond or —OCH₂— and R^{x} and R^{y} are independently hydrogen, C₁₋₆alkyl or combine together to form a ring including piperidinyl, piperazinyl, pyrrolidinyl or morpholinyl group in which a ring is optionally substituted by C₁₋₆alkyl).

Preferably, R¹, R² and R³ are, independently, selected from the group consisting of C₁₋₆alkyl, halogen, C₁₋₆alkoxy, cyano and CF₃.

When m, n or p is other than 0, preferred R¹, R² and R³ groups respectively include C₁₋₆alkyl (particularly methyl), halogen (particularly fluoro or chloro) or C₁₋₆alkoxy (particularly methoxy or ethoxy).

When m, n or p is 2 or 3, the groups R¹, R² and R³ respectively can be the same or different.

Preferably, V is O.

Preferably the ring containing W, X, Y, Z is

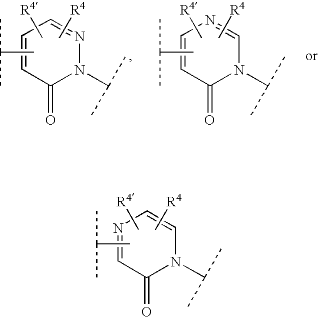

in which the ring nitrogen adjacent to the carbonyl is bonded to the group L.

Suitably, R⁴ and R^{4′} are independently hydrogen, C₁₋₆alkyl or halogen. Preferably R⁴ is hydrogen.

Suitably, L is —(CH₂)_{q}— where q is 0, 1, 2 or 3. Preferably, L is —CH₂—.

### Suitably, J is

(i) a group —CR⁵═CR⁶— where R⁵ and R⁶ are independently hydrogen or C₁₋₆alkyl;
(ii) a group —CHR⁷—CHR⁸— where R⁷ and R⁸ are independently hydrogen, C₁₋₆alkyl, C₃₋₇cycloalkyl, phenyl, naphthyl, thienyl, furyl, pyrrolyl, triazolyl, imidazolyl, oxazolyl, thiazolyl, oxadiazolyl, isothiazolyl, isoxazolyl, thiadiazolyl, pyridonyl, pyrazolyl, pyrimidinyl, pyridazinyl, pyrazinyl, pyridyl quinolyl, isoquinolyl, indolyl, benzofuranyl, benzothienyl, benzimidazolyl, benzoxazolyl, a group —NHCOR⁹ or —NHSO₂R⁹ in which R⁹ is as defined above or a group —(CH₂)₁₋₆NR^{x}R^{y} in which R^{x} and R^{y} are as defined above;
(iii) a single bond;
(iv) —CHR⁶— where R⁶ is as defined above;
(v) a group —O—CHR¹⁰—, —NR¹¹—CHR¹⁰— or —CR¹²R¹³CHR¹⁰— where R¹⁰ and R¹¹ are independently hydrogen or C₁₋₆alkyl and R¹² and R¹³ are independently C₁₋₆alkyl or R¹² and R¹³ combine together to form phenyl, C₃₋₇ cycloalkyl, piperidyl, piperazinyl, pyrrolidinyl or morpholinyl.

### Preferably J is

(i) a group —CR⁵═CR⁶— where R⁵ and R⁶ are independently hydrogen or C₁₋₆alkyl; or
(ii) a group —CHR⁷—CHR⁸— where R⁷ and R⁸ are independently hydrogen, C₁₋₆alkyl, a group —NHCOR⁹ or —NHSO₂R⁹ in which R⁹ is as defined above.

Most preferably, J is selected from the group consisting of —CH═CH—, —(CH₂)₂— and —CHR⁷—CH₂— in which R⁷ is C₁₋₆alkyl (particularly methyl or ethyl).

Particularly preferred compounds of this invention are selected from the group consisting of E1-E18 (as described below) or a pharmaceutically acceptable derivative thereof.

It will be appreciated that the compounds of formula (I) may have one or more asymmetric carbon atoms and therefore may occur as racemates, racemic mixtures and as individual enantiomers or diastereomers. All such isomeric forms are included within the present invention, including mixtures thereof.

Where a compound of the invention contains an alkenyl or alkenylene group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or HPLC. A single stereoisomeric form of the compound may also be prepared from a corresponding optically pure intermediate or by resolution, such as HPLC of the corresponding racemate using a suitable chiral support or by fractional crystallisation of the diastereoisomeric salts formed by reaction of the corresponding racemate with a suitable optically active acid or base, as appropriate. Alternatively a mixture of enantiomers may be separated by chemical reaction with an appropriate chiral compound with the formation of a new covalently bonded species, for example the coupling of a racemic carboxylic acid with a chiral amine or alcohol to give a diastereomeric mixture (in the case of amides or esters respectively), which may be separated by conventional techniques such as column chromatography, HPLC or fractional crystallisation. The single diastereomers may then be converted to the single enantiomers of the desired compound by appropriate chemistry such as hydrolytic cleavage of the new covalent bond.

As used herein, the term “pharmaceutically acceptable derivative”, means any pharmaceutically acceptable salt, solvate or prodrug e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release the compound of formula (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy or amine groups are bonded to any group that, when administered to a patient, cleaves to form the hydroxy or amine groups. Thus, representative examples of prodrugs include (but are not limited to) acetate, formate and benzoate derivatives of alcohol and amine functional groups of the compounds of formula (I). Further, in the case of a carboxylic acid (—COOH), esters may be employed, such as methyl esters, ethyl esters, double esters and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent.

Pharmaceutically acceptable acid salts are formed from acids which form non-toxic salts and examples are hydrochloride, hydrobromide, hydroiodide, sulfate, bisulfate, nitrate, phosphate, hydrogen phosphate, acetate, maleate, malate, fumarate, lactate, tartrate, citrate, formate, gluconate, succinate, piruvate, oxalate, oxaloacetate, trifluoroacetate, saccharate, benzoate, methanesulfonate, ethanesulfonate, benzenesulfonate and p-toluenesulfonate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

In a further aspect, the present invention also provides a process for the preparation of the compound of formula (I) or a pharmaceutically acceptable derivative thereof which comprises hydrolysis of a carboxylic acid ester derivative of formula (II):

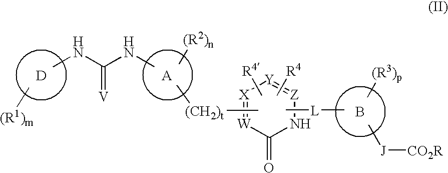

in which R¹-R⁴, R^{4′}, m, n, p, t, A, B, D, L, J, V, W, X, Y and Z are as defined in formula (I) and R is a group capable of forming a carboxylic acid ester and optionally thereafter forming a pharmaceutically acceptable derivative thereof.

An example of a suitable R group is C₁₋₆alkyl such as methyl or t-butyl. Hydrolysis may either occur via an acidic or an alkaline medium. Such methods are familiar to those skilled in the art.

The compounds of formula (II) can be prepared by either:

(a) reacting a compound of formula (III):

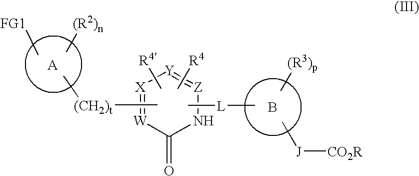

in which R²-R⁴, R^{4′}, n, p, t, A, B, L, J, R, W, X, Y and Z are as defined in formulae (I) or (II) with a compound of formula (IV):

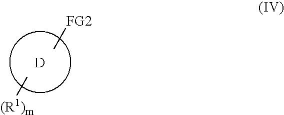

in which R¹, m and D are as defined in formula (I) and FG1 and FG2 contain appropriate functional groups which are capable of reacting together to form the urea moiety; or
(b) reacting the compound of formula (V):

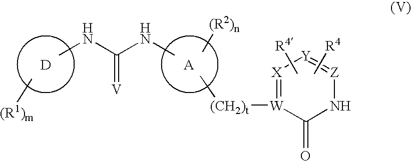

in which R¹, R², R⁴, R^{4′}, m, n, t, A, D, V, W, X, Y and Z are as defined in formula (I) or (II) with a compound of formula (VI):

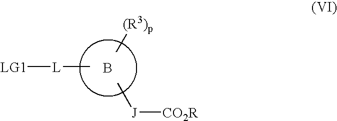

in which p, R, R³, J, B and L are as defined in formulae (I) or (II) and LG1 is a leaving group.

For process (a), suitable examples of appropriate FG1 and FG2 groups include:

### (i) FG1 is —N═C═O and FG2 is NH₂; or FG1 is NH₂ and FG2 is N═C═O; or

(ii) FG1 is NH₂ and FG2 is NH₂ together with an appropriate urea forming agent.

In process (i), the reaction is typically carried out in an inert solvent such as dichloromethane or acetonitrile at ambient temperature.

In process (ii), the reaction is typically carried out in the presence of an appropriate urea forming agent, such as carbonyl diimidazole or phosgene, a suitable solvent being an inert organic solvent such as N,N-dimethylformamide, tetrahydrofuran, or dichloromethane at ambient or elevated temperature optionally in the presence of a base such as triethylamine or pyridine.

For process (b), a suitable example of a leaving group is halogen (particularly chloro) or mesylate. The reaction is typically carried out in an inert solvent such as tetrahydrofuran, N,N-dimethyl formamide or acetonitrile at ambient temperature.

Intermediate compound of formula (II) is believed to be novel and form a yet further aspect of this invention.

Intermediate compounds of formulae (III), (IV), (V) and (VI) are either commercially available or can be prepared using methods described herein, by methods known to those skilled in the art or by analogous methods thereto.

Those skilled in the art will appreciate that in the preparation of the compound of the invention it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & Sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl). Examples of suitable oxygen protecting groups may include alkyl silyl groups, such as trimethylsilyl or tert-butyldimethylsilyl; alkyl ethers such as tetrahydropyranyl or tert-butyl; or esters such as acetate.

Compounds of this invention may be tested for in vitro biological activity in accordance with the following assay.

### Jurkat J6 Scintillation Proximity Assay (SPA)

The Jurkat J6 Scintillation Proximity Assay was used to investigate the interaction of the integrin VLA-4 expressed on the Jurkat J6 cell membrane with test compounds. J6 cells (1 million cells/well) were allowed to coat wheat germ agglutinin coated SPA beads (Amersham, 1 mg/well) in assay buffer containing 50 mM HEPES, 100 mM NaCl and 1 mM MnCl₂ (pH adjusted to 7.5 with 4M NaOH). Tritiated ³H Standard Compound A (1-3 nM final assay concentration) and test compounds were dissolved in an appropriate solvent and diluted in assay buffer (the top assay concentration being 2.5 μm; ten point dose response curve). Compounds were assayed in duplicate, a four parameter curve fit being applied. The equilibrium dissociation constant for each compound was calculated according to the method of Cheng & Prusoff (Biochem Pharmacol., 22(23): 3099-3108 (1973)). Data were presented as the mean pKi.

Standard compound A is (2S)-3-[4-({[4-(aminocarbonyl)-1-piperidinyl]carbonyl}oxy)-phenyl]-2-[((2S)-4-methyl-2-{[2-(2-methylphenoxy)acetyl]amino}pentanoyl)-amino]propanoic acid potassium salt which is described in patent application WO 00/37444 (Glaxo Group Ltd. et al.). Tritiated ³H derivatives may be prepared employing conventional methods.

All examples prepared in accordance with this invention were tested in accordance with this procedure and were found to have a pKi ≧8.0.

Compounds of formula (I) or a pharmaceutically acceptable derivatives thereof inhibit α₄ integrin mediated cell adhesion. It is believed that α₄ integrin mediated cell adhesion is implicated in a range of conditions such as rheumatoid arthritis (RA); asthma; allergic conditions such as rhinitis; adult respiratory distress syndrome; AIDS-dementia; Alzheimer's disease; cardiovascular diseases; thrombosis or harmful platelet aggregation; reocclusion following thrombolysis; reperfusion injury; skin inflammatory diseases such as psoriasis, eczema, contact dermatitis and atopic dermatitis; diabetes (e.g., insulin-dependent diabetes mellitus, autoimmune diabetes); multiple sclerosis; systemic lupus erythematosus (SLE); inflammatory bowel disease such as ulcerative colitis, Crohn's disease (regional enteritis) and pouchitis (for example, resulting after proctocolectomy and ileoanal anastomosis); diseases associated with leukocyte infiltration to the gastrointestinal tract such as Celiac disease, nontropical Sprue, enteropathy associated with seronegative arthropathies, lymphocytic or collagenous colitis, and eosinophilic gastroenteritis; diseases associated with leukocyte infiltration to other epithelial lined tissues, such as skin, urinary tract, respiratory airway, and joint synovium; pancreatitis; mastitis (mammary gland); hepatitis; cholecystitis; cholangitis or pericholangitis (bile duct and surrounding tissue of the liver); bronchitis; sinusitis; inflammatory diseases of the lung which result in interstitial fibrosis, such as hypersensitivity pneumonitis; collagen disease (in SLE and RA); sarcoidosis; osteoporosis; osteoarthritis; atherosclerosis; neoplastic diseases including metastasis of neoplastic or cancerous growth; wound healing enhancement; certain eye diseases such as retinal detachment, allergic conjunctivitis and autoimmune uveitis; Sjogren's syndrome; rejection (chronic and acute) after organ transplantation; host vs. graft or graft vs. host diseases; intimal hyperplasia; arteriosclerosis (including graft arteriosclerosis after transplantation); reinfarction or restenosis after surgery such as percutaneous transluminal coronary angioplasty (PTCA) and percutaneous transluminal artery recanalization; nephritis; tumor angiogenesis; malignant tumor; multiple myeloma and myeloma-induced bone resorption; sepsis; and central nervous system injury such as stroke, traumatic brain injury and spinal cord injury and Meniere's disease.

The compounds of the present invention can be preferably used for the treatment or prevention of asthma, allergic conditions such as rhinitis, inflammatory bowel disease such as ulcerative colitis and Crohn's disease, rheumatoid arthritis, atopic dermatitis, multiple sclerosis and rejection after organ transplantation.

The present invention further provides a method for the treatment or prevention of conditions in which an inhibitor of α₄ integrin mediated cell adhesion is beneficial which comprises administering to a patient in need thereof a safe and effective amount of the compound of formula (I) or a pharmaceutically acceptable derivative thereof. The present invention especially provides a method for the treatment or prevention of the aforementioned conditions.

The present invention also provides the compound of formula (I) or a pharmaceutically acceptable derivative thereof for use in therapy, particularly the treatment or prevention of the aforementioned disorders.

In another aspect, the invention provides a use of the compound of formula (I) or a pharmaceutically acceptable derivative thereof in the manufacture of a medicament for the treatment or prevention of conditions in which an inhibitor of α₄ integrin mediated cell adhesion is beneficial, particularly the aforementioned disorders.

While it is possible for the compounds of the present invention to be administered alone, it is preferable to formulate into a pharmaceutical composition in accordance with standard pharmaceutical practice. Thus the invention also provides a pharmaceutical composition which comprises a therapeutically effective amount of the compound of formula (I) or a pharmaceutically acceptable derivative thereof in admixture with a pharmaceutically acceptable carrier or diluent.

The invention further provided a pharmaceutical composition comprising the compound of formula (I) or a pharmaceutically acceptable derivative thereof together with another therapeutically active agent.

There is further provided by the present invention a process of preparing a pharmaceutical composition, which process comprises mixing at least one compound of the invention, together with a pharmaceutically acceptable carrier or diluent.

The pharmaceutical compositions may be for human or animal usage in human and veterinary medicine and will typically comprise any one or more of a pharmaceutically acceptable diluent, carrier or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The carrier or diluent must be acceptable in the sense of being not deleterious to the recipient thereof. The pharmaceutically acceptable carrier or diluent may be, for example, binders (e.g., syrup, gum arabic, gelatin, sorbitol, tragacanth, polyvinylpyrrolidone), excipients (e.g., lactose, sucrose, corn starch, potassium phosphate, sorbitol, glycine), lubricants (e.g., magnesium stearate, talc, polyethylene glycol, silica) disintegrators (e.g., potato starch), wetting agents (e.g., sodium laurylsulfate), and the like.

The routes for administration (delivery) of the composition of the invention include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural, sublingual.

For example, the compound can be administered orally in the form of tablets, capsules, ovules, elixirs, solutions or suspensions, which may contain flavouring or colouring agents, for immediate-, delayed-, modified-, sustained-, pulsed- or controlled-release applications. The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example, see International Patent Application No. WO 02/00196 (SmithKline Beecham).

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA) (for example, from Ineos Fluor), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compound and a suitable powder base such as lactose or starch.

Alternatively, the compound of the present invention can be administered in the form of a suppository or pessary, or it may be applied topically in the form of a gel, hydrogel, lotion, solution, cream, ointment or dusting powder. The compound of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection.

In a preferred embodiment, the agents of the present invention are delivered systemically (such as orally, buccally, sublingually), more preferably orally.

Hence, preferably the agent is in a form that is suitable for oral delivery.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses.

A proposed dose of the compounds according to the present invention for administration to a human (of approximately 70 kg body weight) is 0.1 mg to 2 g, more typically 1 mg to 500 mg of the active ingredient per unit dose, expressed as the weight of free base. The unit dose may be administered, for example, 1 to 4 times per day. The dose will depend on the route of administration. It will be appreciated that it may be necessary to make routine variations to the dosage depending on the age and weight of the patient as well as the severity of the condition to be treated. The dosage will also depend on the route of administration. The precise dose and route of administration will ultimately be at the discretion of the attendant physician or veterinarian.

The compounds of the invention may also be used in combination with other therapeutic agents. The invention thus provides, in a further aspect, a combination comprising a compound of the invention together with a further therapeutic agent.

When a compound of the invention is used in combination with a second therapeutic agent active against the same disease state the dose of each compound may differ from that when the compound is used alone. Appropriate doses will be readily appreciated by those skilled in the art. It will be appreciated that the amount of the compound of the invention required for use in treatment will vary with the nature of the condition being treated and the age and the condition of the patient and will be ultimately at the discretion of the attendant physician or veterinarian. Examples of other active agents that may be combined with the compound of the invention include, but not limited to: (a) other VLA-4 antagonists; (b) H1 histamine antagonists; (c) NSAID's; (d) anti-diabetic agents e.g. glitazones (e) anti-cholinergic agents (f) COX-2 inhibitors; (g) PDE-IV inhibitors; (h) steroids e.g. corticosteroids; (i) beta agonists; (j) antagonists of the chemokine receptors e.g. CCR-2, CCR-3, CCR-5 and CCR-8; (k) suitable multiple sclerosis agents such as beta interferons; and (l) LFA-1 antagonists; (m) TNF inhibitors; (n) Sulphasalazine and 5-aminosalicylates and (O) Immunosuppressants.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route. When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art. All publications, including but not limited to patents and patent applications, cited in this specification are herein incorporated by reference as if each individual publication were specifically and individually indicated to be incorporated by reference herein as though fully set forth.

The following Preparations and Examples illustrate the preparation of compounds of the invention. All reactions were carried out at ambient temperature unless otherwise specified.

### Preparation 1

### 3-(4-Hydroxymethylphenyl)acrylic acid ethyl ester (P1)

4-Bromobenzyl alcohol (10.5 g, 56.1 mmol), triphenylphosphine (0.5 g, 1.9 mmol) and palladium acetate (0.5 g, 2.2 mmol) were stirred at reflux in ethyl acrylate (20 mL) and triethylamine (100 mL) for 72 hours, then allowed to cool. The reaction mixture was filtered through Celite (Diatomaceous Earth), then concentrated. The crude solid was purified by chromatography on silica gel (20% v/v ethyl acetate in petroleum ether) to afford the title compound as an oil.

### Preparation 2

### 3-(4-Hydroxymethylphenyl)propionic acid ethyl ester (P2)

3-(4-Hydroxymethylphenyl)acrylic acid ethyl ester (P1, 3 g, 14.5 mmol) and palladium on charcoal (0.3 g) in ethanol (30 mL) was stirred for 4 hours under atmospheric pressure of hydrogen. The reaction mixture was filtered through Celite (Diatomaceous Earth) and concentrated to afford the title compound as an oil.

### Preparation 3

### 3-(4-Chloromethylphenyl)propionic acid ethyl ester (P3)

To a stirred solution of 3-(4-hydroxymethylphenyl)propionic acid ethyl ester (P2, 2.9 g, 13.9 mmol) in triethylamine (4.0 mL, 27.8 mmol) and dichloromethane (30 mL) was slowly added at 0° C. methanesulfonyl chloride (1.6 mL, 20.9 mmol). The solution was stirred at room temperature for 18 hours, then the solution was washed with 1N aqueous hydrochloric acid. The organic phase was dried (anhydrous magnesium sulfate) and concentrated to afford the title compound as an oil.

### Preparation 4

### (E)-3-(4-Formylphenyl)but-2-enoic acid ethyl ester (P4)

4-Bromobenzaldehyde (12.0 g, 65 mmol), ethyl crotonate (26.0 mL, 209 mmol), triphenylphosphine (0.5 g, 2 mmol) and palladium (II) acetate (0.5 g, 2 mmol) were stirred at reflux under argon for 24 hours. The mixture was then filtered and concentrated in vacuo to yield a dark brown oil. This was purified by chromatography on silica gel (0-30% ethyl acetate in hexane, gradient), giving the title compound as an oil; MS (ES+ve): [M+H]⁺ at m/z 219 (C₁₃H₁₄O₃ requires [M+H]⁺ at m/z 219).

### Preparation 5

### (R,S)-3-(4-Hydroxymethylphenyl)butyric acid ethyl ester (P5)

A mixture of (E)-3-(4-formylphenyl)but-2-enoic acid ethyl ester (P4, 8.74 g, 40 mmol) and 10% palladium on carbon (60% aqueous paste, 0.5 g) was hydrogenated in ethanol (200 mL) at atmospheric pressure for 4 hours. The mixture was filtered through kieselguhr, and the filtrate then concentrated in vacuo to give a colourless oil. After purification by chromatography on silica gel (0-60% ethyl acetate in hexane, gradient) the title compound was obtained as a colourless oil; MS (ES+ve): [M-OH]⁺ at m/z 205 (C₁₃H₁₈O₃ requires [M-OH]⁺ at m/z 205).

### Preparation 6

### 4-[(S)-2-((S)-2-Hydroxy-1-phenylethylcarbamoyl)-1-methylethyl]benzoic acid ethyl ester (P6a) and 4-[(R)-2-((S)-2-hydroxy-1-phenylethylcarbamoyl)-1-methylethyl]-benzoic acid ethyl ester (P6b)

To a solution of 4-(2-carboxy-1-methylethyl)benzoic acid ethyl ester (J. I. DeGraw et al. J. Med. Chem. 1986, 29, 1056) (3.54 g, 15 mmol) in dichloromethane (100 mL) cooled in an ice bath was added oxalyl chloride (3.9 mL, 45 mmol). N,N-Dimethylformamide (0.1 mL) was added and the mixture stirred at room temperature for 2 hours, then concentrated under reduced pressure. The residual acid chloride was dissolved in dichloromethane (60 mL) and added to an ice-cooled mixture of (S)-2-phenylglycinol (2.72 g, 20 mmol) and triethylamine (6.3 mL, 45 mmol) in dichloromethane (60 mL) over 30 minutes. The reaction mixture was stirred at room temperature for 1 hour. 2N Hydrochloric acid was added, the organic phase was separated, then washed with water, dried (anhydrous magnesium sulfate) and evaporated. The diastereomeric products were separated by flash chromatography with elution by ethyl acetate, then ethyl acetate-methanol (9:1). There were obtained an earlier eluting diastereomer (P6a); TLC (silica gel; ethyl acetate) R_{f} 0.36; MS (ES+ve): [M+H]⁺ at m/z 356 (C₂₁H₂₅NO₄ requires [M+H]⁺ at m/z 356); and a later eluting diastereomer (P6b); TLC (silica gel; ethyl acetate) R_{f} 0.19; MS (ES+ve): [M+H]⁺ at m/z 356 (C₂₁H₂₅NO₄ requires [M+H]⁺ at m/z 356)

### Preparation 7

### (S)-3-(4-Hydroxymethylphenyl)-N—((S)-2-hydroxy-1-phenylethyl)butyramide (P7a) and (R)-3-(4-hydroxymethylphenyl)-N—((S)-2-hydroxy-1-phenylethyl)butyramide (P7b)

To a solution of the later eluting diastereomer, 4-[(R)-2-((S)-2-hydroxy-1-phenylethylcarbamoyl)-1-methylethyl]benzoic acid ethyl ester, (P6b, 2.42 g, 6.81 mmol) in tetrahydrofuran (100 mL) was added a solution of lithium borohydride in tetrahydrofuran (2M, 15 mL, 30 mmol). Methanol (1 mL) was added dropwise and the reaction mixture stirred at room temperature for 2 hours. A further portion of lithium borohydride in tetrahydrofuran (2M, 10 mL, 20 mmol) and methanol (0.8 mL) were added and the mixture stirred for a further 3 hours, then cooled in an ice bath. 2N Hydrochloric acid (100 mL) was added cautiously, then the mixture was concentrated under reduced pressure. Ethyl acetate was added and the organic phase washed with water, then brine, dried (anhydrous magnesium sulfate) and evaporated to give one diastereomer (P7b) of the title compound; MS (ES-ve): [M−H]⁻ at m/z 312 (C₁₉H₂₃NO₃ requires [M−H]⁻ at m/z 312).

The other diastereomer (P7a) was prepared in a similar manner from the earlier eluting diastereomer 4-[(S)-2-((S)-2-hydroxy-1-phenylethylcarbamoyl)-1-methylethyl]benzoic acid ethyl ester (P6a).

### Preparation 8

### (R)-(−)-3-(4-Hydroxymethylphenyl)butyric acid methyl ester (P8)

To a solution of the diastereomer (R)-3-(4-hydroxymethylphenyl)-N—((S)-2-hydroxy-1-phenylethyl)butyramide (P7b, 2.0 g, 6.38 mmol) in dioxane (85 mL) was added 3N sulphuric acid (85 mL). The mixture was heated at reflux for 6 hours, cooled and then concentrated under reduced pressure. The concentrate was extracted three times with ethyl acetate, the combined organic phases were washed with water, then brine, dried (anhydrous magnesium sulfate) and evaporated. The residual solid was dissolved in methanol (90 mL) and concentrated sulphuric acid (2 mL) added. The mixture was refluxed for 1 hour, cooled and then concentrated under reduced pressure. Water and ethyl acetate were added and the organic phase was washed with water, then brine, dried (anhydrous magnesium sulfate) and evaporated. Purification by flash chromatography with elution by ethyl acetate-hexane (1:1) gave the title compound as a colourless oil; [α]_{D}^{30° C.}−41.2° (c=1.0, MeOH).

### Preparation 9

### (S)-(+)-3-(4-Hydroxymethylphenyl)butyric acid methyl ester (P9)

The title compound was prepared from the diastereomer P7a in a similar manner to that of Preparation 8; [α]_{D}^{30° C.}+42.4° (c=1.0, MeOH).

### Preparation 10

### (R)-3-(4-Methanesulfonyloxymethylphenyl)butyric acid methyl ester (P10)

A solution of (R)-(−)-3-(4-hydroxymethylphenyl)butyric acid methyl ester (P8, 400 mg, 1.80 mmol) in dichloromethane (10 mL) was cooled in an ice bath and treated with triethylamine (0.28 mL, 1.99 mmol) and methanesulfonyl chloride (0.15 mL, 1.99 mmol). The reaction was stirred in an ice bath for 1 hour and then the mixture was diluted with dichloromethane and water. After separation of the organic layer the aqueous phase was further extracted with dichloromethane and then the combined organic layers dried over magnesium sulfate. After filtration and evaporation of the solvent the resulting crude product was used directly without further purification.

Preparations 6-10 are represented in the following reaction scheme.

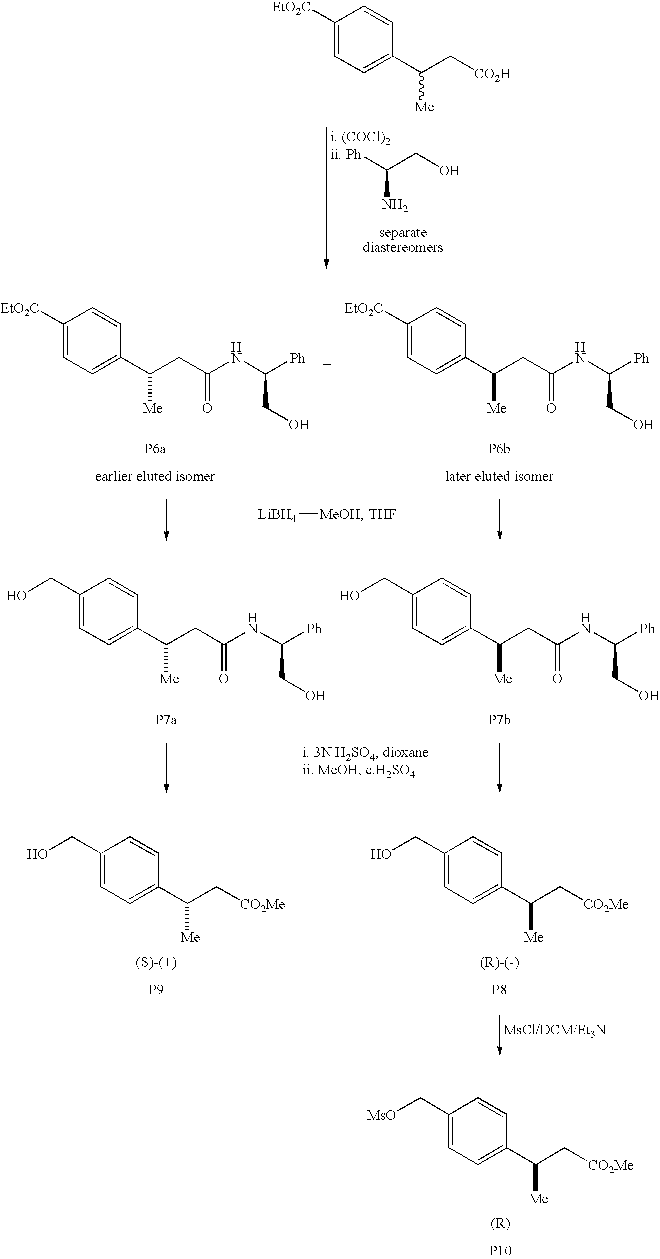

### Preparation 11

### 2-Ethoxy-4-methyl-1-nitrobenzene (P11)

Sodium hydride (60% in mineral oil, 2.30 g, 57 mmol) was stirred under argon in solid carbon dioxide bath cooled N,N-dimethylformamide (100 mL) as 2-hydroxy-4-methyl-1-nitrobenzene (8.00 g, 52 mmol) was added in dry N,N-dimethylformamide (75 mL) over 10 minutes. The mixture was stirred at ambient temperature for 1 hour, re-cooled in ice, and treated with iodoethane (4.6 mL, 57 mmol). This mixture was stirred at ambient temperature for 5 days, concentrated at reduced pressure, diluted with ethyl acetate (200 mL), washed with water (3×200 mL) and brine (200 mL), dried over magnesium sulfate, filtered and concentrated at reduced pressure to yield the title compound. This material was used in the next step without further purification; LC/MS (ES+ve): [M+H]⁺ at m/z 182 (C₉H₁₁NO₃ requires [M+H]⁺ at m/z 182).

### Preparation 12

### [(E)-2-(3-Ethoxy nitrophenyl)vinyl]dimethylamine (P12)

2-Ethoxy-4-methyl-1-nitrobenzene (P11, 9.26 g, 51 mmol) and tert-butoxybis(dimethylamino)methane (20.2 mL, 98 mmol) were heated to 100° C. for 16 hours, and then concentrated at reduced pressure to yield the title compound. This material was used in the next step without further purification; LC/MS (ES-ve): M⁻ at m/z 236 (C₁₂H₁₆N₂O₃ requires M⁻ at m/z 236).

### Preparation 13

### (3-Ethoxy-4-nitrophenyl)acetonitrile (P13)

[(E)-2-(3-Ethoxy-4-nitrophenyl)vinyl]dimethylamine (P12, 12.10 g, 51 mmol) and hydroxylamine-O-sulfonic acid (17.40 g, 154 mmol) were stirred under argon in water (200 mL) for 5 hours. The title compound was obtained as a solid after filtration and drying. This material was used in the next step without further purification; LC/MS (ES-ve): [M−H]⁻ at m/z 205 (C₁₀H₁₀N₂O₃ requires [M−H]⁻ at m/z 205).

### Preparation 14

### 2-(3-Ethoxy nitrophenyl)acetamide (P14)

(3-Ethoxy-4-nitrophenyl)acetonitrile (P13, 5.0 g, 190 mmol) was stirred vigorously in concentrated hydrochloric acid (20 mL) for 48 hours. It was then diluted with water (100 mL), and extracted into ethyl acetate (3×100 mL). The organic phase was washed with saturated sodium bicarbonate (2×100 mL), dried over magnesium sulfate, filtered and concentrated at reduced pressure to yield the title compound. This material was used in the next step without further purification; LC/MS (ES+ve): [M+H]⁺ at m/z 225 (C₁₀H₁₂N₂O₄ requires [M+H]⁺ at m/z 225).

### Preparation 15

### 5-(3-Ethoxy-4-nitrophenyl)-3H-pyrimidin-one (P15)

2-(3-Ethoxy-4-nitrophenyl)acetamide (P14, 1.78 g, 8.0 mmol) and N,N′,N″-methylidynetrisformamide (2.30 g, 16 mmol) were stirred under argon in formamide (3 mL). The mixture was heated to 160° C. for 8 hours, diluted with water (25 mL) and treated with 2N aqueous sodium hydroxide (10 mL). It was heated in a steam bath until dissolution occurred, and then treated with charcoal (2.5 g), sonicated and then filtered. Carbon dioxide was bubbled into the filtrate until pH 7 was achieved. The resulting precipitate was filtered, then azeotroped with toluene (3×50 mL) to yield the title compound. This material was used in the next step without further purification; LC/MS (ES+ve): [M+H]⁺ at m/z 262 (C₁₂H₁₁N₃O₄ requires [M+H]⁺ at m/z 262).

### Preparation 16

### 5-(4-Amino-3-ethoxyphenyl)-3H-pyrimidin-4-one (P16)

5-(3-Ethoxy-4-nitrophenyl)-3H-pyrimidin-4-one (P15, 830 mg, 3.2 mmol) was stirred under argon in ethyl acetate (50 mL) and ethanol (50 mL). Tin (II) chloride dihydrate (3.59 g, 16 mmol) was added and the mixture was heated at 80° C. for 5 hours. Saturated sodium bicarbonate (100 mL) was added and the mixture was filtered, the filtrate was extracted into ethyl acetate (3×50 mL), washed with water (2×50 mL) and brine (50 mL), dried over magnesium sulfate, filtered and concentrated at reduced pressure to yield the title compound. This material was used in the next step without further purification; LC/MS (ES+ve): [M+H]⁺ at m/z 232 (C₁₂H₁₃N₃O₂ requires [M+H]⁺ at m/z 232).

### Preparation 17

### 1-[2-Ethoxy-4-(6-oxo-1,6-dihydropyrimidin-5-yl)phenyl]-3-o-tolylurea (P17)

5-(4-Amino-3-ethoxyphenyl)-3H-pyrimidin-4-one (P16, 390 mg, 1.7 mmol) was stirred under argon in dichloromethane (20 mL). o-Tolylisocyanate (0.32 mL, 2.5 mmol) was added over 5 minutes, the reaction was stirred for a further 4 hours and then concentrated at reduced pressure to yield the title compound as a solid. This material was used in the next step without further purification; LC/MS (ES+ve): [M+H]⁺ at m/z 365 (C₂₀H₂₀N₄O₃ requires [M+H]⁺ at m/z 365).

### Preparation 18

### (R,S)-3-(4{(5-[3-Ethoxy-4-(tolylureido)phenyl]-6-oxo-6H-pyrimidin-1-ylmethyl}-phenyl)butyric acid ethyl ester (P18)

(R,S)-3-(4-Methanesulfonyloxymethylphenyl)butyric acid ethyl ester (prepared from (R,S)-3-(4-hydroxymethylphenyl)butyric acid ethyl ester (P5) by a method analogous to that described in Preparation 10, 495 mg, 1.5 mmol) was stirred under argon in N,N-dimethylformamide (20 mL). 1-[2-Ethoxy-4-(6-oxo-1,6-dihydropyrimidin-5-yl)phenyl]-3-o-tolylurea (P17, 400 mg, 1.1 mmol) and cesium carbonate (716 mg, 2.2 mmol) were added and the mixture was stirred for 16 hours. The mixture was diluted with ethyl acetate (20 mL) and water (20 mL) and after separation of the organic layer, the aqueous phase was re-extracted with ethyl acetate (2×20 mL). The combined organic layers were washed with brine (20 mL), dried over magnesium sulfate, filtered and concentrated at reduced pressure. The crude product was purified by silica gel chromatography (Flashmaster II, 50 g silica) eluting with ethyl acetate:hexane (66:34) to yield the title compound as a colourless solid; LC/MS (AP+ve): [M+H]⁺ at m/z 569 (C₃₃H₃₆N₄O₅ requires [M+H]⁺ at m/z 569).

Preparations 11-18 are represented by the following reaction scheme.

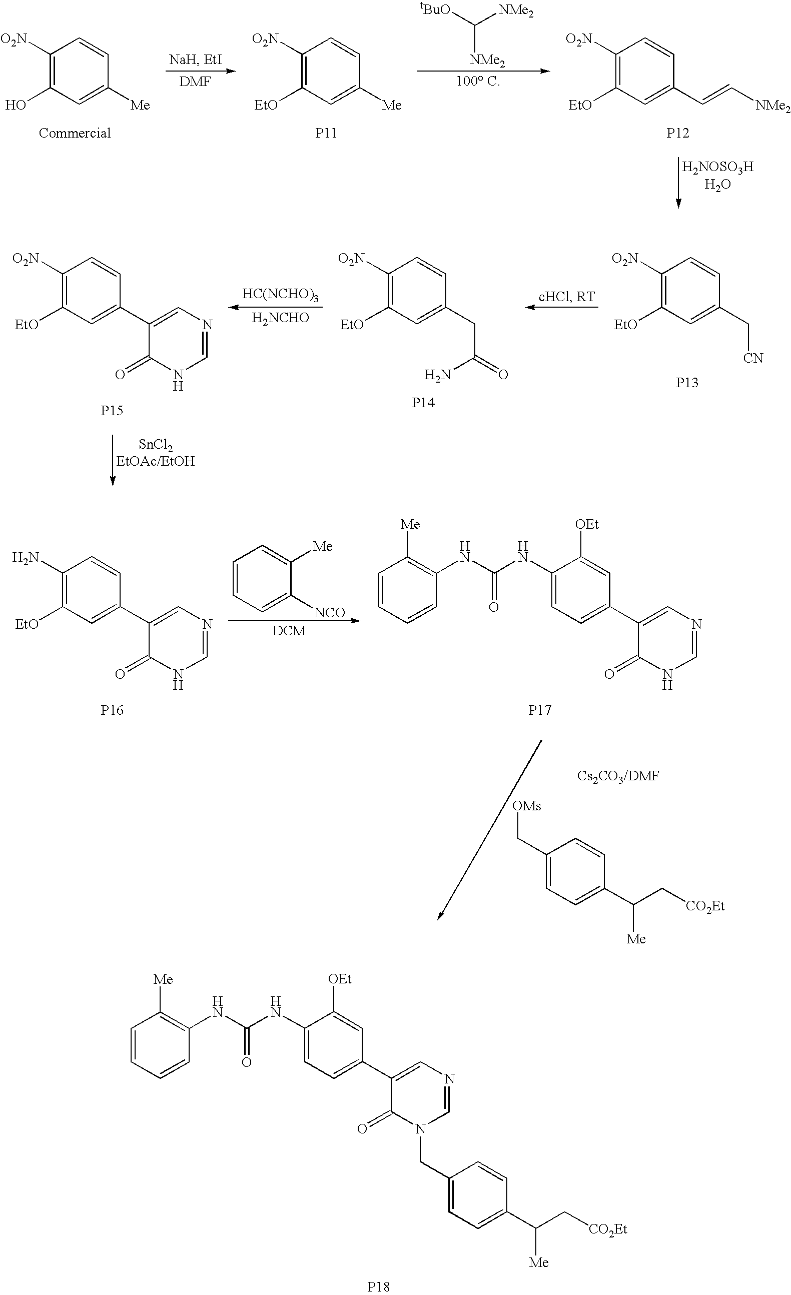

### Preparation 19

### (R)-3-{4-[(4-Nitrophenyl)-6-oxo-6H-pyrimidin-1-ylmethyl]phenyl}butyric acid methyl ester (P19)

To a solution of 5-(4-nitrophenyl)-3H-pyrimidinone (400 mg, 1.8 mmol) {Tsatsaronis et al., Chem. Ber., 94, 1961, 2876} in N,N-dimethylformamide (7 mL) was added cesium carbonate (1.2 g, 3.6 mmol) followed by a solution of (R)-3-(4-methanesulfonyloxymethylphenyl)butyric acid methyl ester (P10, ˜1.8 mmol) in N,N-dimethylformamide (3 mL). The reaction was stirred at room temperature for 2 hours and then diluted with ethyl acetate. After separation of the organic layer the aqueous phase was further extracted with ethyl acetate. The combined organic layers were dried over magnesium sulfate, filtered and concentrated in vacuo. The crude product was purified by chromatography on silica gel eluting with 60:40 ethyl acetate:hexane to yield the title compound as a solid; MS (APCI+ve): [M+H]⁺ at m/z 408 (C₂₂H₂₁N₃O₅ requires [M+H]⁺ at m/z 408).

### Preparation 20

### (R)-3-{4-[5-(4-Aminophenyl)-6-oxo-6H-pyrimidin-1-ylmethyl]phenyl}butyric acid methyl ester (P20)

To a solution of (R)-3-{4-[5-(4-nitrophenyl)-6-oxo-6H-pyrimidin-1-ylmethyl]phenyl}butyric acid methyl ester (P19, 420 mg, 1.03 mmol) in 1:1 ethyl acetate:ethanol (30 mL) was added tin (II) chloride dihydrate (1.2 g, 5.30 mmol). The reaction mixture was heated at 80° C. for 2 hours and then allowed to cool to room temperature. Saturated aqueous sodium hydrogen carbonate (20 mL) was added and the resulting precipitate removed by filtration. The product was extracted into ethyl acetate, dried over magnesium sulfate, filtered and concentrated in vacuo. The residue was purified by chromatography on silica gel eluting with 70:30 ethyl acetate:hexane to yield the title compound as a foam; MS (APCI+ve): [M+H]⁺ at m/z 378 (C₂₂H₂₃N₃O₃ requires [M+H]⁺ at m/z 378).

### Preparation 21

### (R)-3-(4-{6-Oxo-5-[4-(3-tolylureido)phenyl]-6H-pyrimidin-1-ylmethyl}phenyl)butyric acid methyl ester (P21)

To a solution of (R)-3-{4-[5-(4-aminophenyl)-6-oxo-6H-pyrimidin-1-ylmethyl]phenyl}butyric acid methyl ester (P20, 360 mg, 0.95 mmol) in dichloromethane (10 mL) was added o-tolylisocyanate (0.18 mL, 1.45 mmol). The reaction was stirred at room temperature for 14 hours and then concentrated in vacuo. The crude product was purified by chromatography on silica gel eluting with 70:30 ethyl acetate:hexane to yield the title compound as a colourless foam. MS (ES+ve): [M+H]⁺ at m/z 511 (C₃₀H₃₀N₄O₄ requires [M+H]⁺ at m/z 511).

### Preparation 22

### 3-(4-Aminophenyl)-1H-pyrazin-2-one (P22)

3-Phenyl-1H-pyrazin-2-one (4.3 g, 25 mmol, prepared by the method of G Karmas and P. E. Spoerri, J. Amer. Chem. Soc., 1956, 78, 4071) was added portion-wise with stirring to a mixture of concentrated sulphuric acid (5 mL) and fuming nitric acid (15 mL) pre-cooled to −40° C., keeping the temperature below −30° C. during the addition. The reaction mixture was stirred for a further hour, gradually warming to 0° C., and then poured into stirring ice/water (125 mL). A mixture of nitro isomers was obtained after collection of the resulting solid, washing with water and drying in vacuo. The required 3-(4-nitrophenyl)-1H-pyrazin-2-one isomer can be obtained from this mixture as the first crop by fractional crystallization from acetone. Hydrogenation (10% Pd/C, 50 psi) in ethanol/water afforded the title compound as a solid; MS (AP+ve): [M+H]⁺ at m/z 188 (C₁₀H₉N₃O requires [M+H]⁺ at m/z 188).

Further product was obtained by hydrogenation of the second crop material and separation of the resulting isomers by chromatography on silica gel eluting with 1:3 methanol/sat. ammonia: dichloromethane, the title compound eluting first.

### Preparation 23

### 4-(1-Benzyloxycarbonylmethylenepropyl)benzoic acid methyl ester (P23)

A solution of (dimethoxyphosphoryl)acetic acid benzyl ester (6.7 g, 25.9 mmol) in dry N,N-dimethylformamide (20 mL) was added dropwise with stirring under argon to an ice-bath cooled suspension of sodium hydride (1.1 g, 60% dispersion in oil, 27.5 mmol) in dry N,N-dimethylformamide (60 mL), and then the mixture stirred at room temperature for 30 minutes. A solution of 4-propanoylbenzoic acid methyl ester (5.0 g, 26.0 mmol) in dry N,N-dimethylformamide (20 mL) was added and stirring continued overnight at room temperature. The mixture was concentrated under reduced pressure and then partitioned between ethyl acetate (100 mL) and water containing 10% acetic acid (50 mL). The aqueous layer was further extracted with ethyl acetate (2×100 mL) and the combined organic layers washed with brine (50 mL), dried over anhydrous magnesium sulfate, filtered and evaporated to dryness. Purification by column chromatography on silica gel with a gradient of 15-30% ethyl acetate in hexane gave a mixture of E and Z isomers of the title compound together with the double bond positional isomer 4-((E)-1-benzyloxycarbonylmethylpropenyl)benzoic acid methyl ester. This mixture was carried forward without further purification.

### Preparation 24

### (R,S)-4-(1-Carboxymethylpropyl)benzoic acid methyl ester (P24)

The mixture of double bond isomers including 4-(1-benzyloxycarbonyl-methylenepropyl)benzoic acid methyl ester (P23, 3.37 g, 10.4 mmol) in methanol (150 mL) with 10% palladium on charcoal was hydrogenated at atmospheric pressure and room temperature for 5 hours. After filtration through a pad of Celite and washing with further methanol the resulting solution was evaporated to dryness to give the product initially as a colourless oil that solidified on standing.

### Preparation 25

### 4-(S)-1-[((S)-2-Hydroxy-1-phenylethylcarbamoyl)methyl]propyl)benzoic acid methyl ester (P25a) and 4-{(R)-1-[((S)-2-Hydroxy-1-phenylethylcarbamoyl)methyl]-propyl}benzoic acid methyl ester (P25b)

The title compounds were prepared from (R,S)-4-(1-carboxymethylpropyl)benzoic acid methyl ester (P24) by a procedure analogous to the method of Preparation 6.

The diastereomeric products were separated by column chromatography on silica gel with ethyl acetate and then 5-10% methanol in ethyl acetate as eluent.

Early fractions contained 4-{(S)-1-[((S)-2-hydroxy-1-phenylethylcarbamoyl)methyl]-propyl}benzoic acid methyl ester (P25a) as a white solid; MS (ES+ve): [M+H]⁺ at m/z 356 (C₂, H₂₅NO₄ requires [M+H]⁺ at m/z 356).

Later fractions contained 4-{(R)-1-[((S)-2-hydroxy-1-phenylethylcarbamoyl)methyl]-propyl}benzoic acid methyl ester (P25b) as a white solid; MS (ES+ve): [M+H]⁺ at m/z 356 (C₂₁H₂₅NO₄ requires [M+H]⁺ at m/z 356).

### Preparation 26

### (S)-3-(4-Hydroxymethylphenyl)pentanoic acid ((S)-2-hydroxy-1-phenylethyl) amide (P26a) and (R)-3-(4-Hydroxymethylphenyl)pentanoic acid ((S)-2-hydroxy-1-phenylethyl) amide (P26b)

The later eluting diastereomer 4-{(R)-1-[((S)-2-hydroxy-1-phenylethylcarbamoyl)-methyl]propyl}benzoic acid methyl ester (P25b) was reduced to the title compound (R)-3-(4-hydroxymethylphenyl)pentanoic acid ((S)-2-hydroxy-1-phenylethyl) amide (P26b) with lithium borohydride by the method of Preparation 7; MS (ES-ve): [M−H]⁻ at m/z 326 (C₂₀H₂₅NO₃ requires [M−H]⁻ at m/z 326).

The other diastereomer, (S)-3-(4-hydroxymethylphenyl)pentanoic acid ((S)-2-hydroxy-1-phenylethyl) amide (P26a) was prepared in an analogous fashion from the earlier eluting diastereomer 4-{(S)-1-[((S)-2-hydroxy-1-phenylethylcarbamoyl)methyl]propyl}benzoic acid methyl ester (P25a); MS (ES-ve): [M−H]⁻ at m/z 326 (C₂₀H₂₅NO₃ requires [M−H]⁻ at m/z 326).

### Preparation 27

### (R)-3-(4-Hydroxymethylphenyl)pentanoic acid (P27)

A solution of (R)-3-(4-hydroxymethylphenyl)pentanoic acid ((S)-2-hydroxy-1-phenylethyl) amide (P26b, 2.93 g, 8.24 mmol) in dioxane (120 mL) and 3N sulfuric acid (120 mL) was heated at reflux for 5 hours, cooled and concentrated under reduced pressure. After extraction with ethyl acetate (3×100 mL) the combined organic layers were washed with water (50 mL) followed by brine (50 mL) and then dried over anhydrous magnesium sulfate. The title compound was obtained after filtration and evaporation to dryness.

### Preparation 28

### (S)-3-(4-Hydroxymethylphenyl)pentanoic acid (P28)

The title compound was prepared in the same manner as the corresponding (R) isomer (P27) from (S)-3-(4-hydroxymethylphenyl)pentanoic acid ((S)-2-hydroxy-1-phenylethyl) amide (P26a). MS (ES-ve): [M−H]⁻ at m/z 207 (C₁₂H₁₆NO₃ requires [M−H]⁻ at m/z 207).

### Preparation 29

### (R)-(−)-3-(4-Hydroxymethylphenyl)pentanoic acid methyl ester (P29)

A solution of (R)-3-(4-hydroxymethylphenyl)pentanoic acid (P27, 2.0 g, 9.6 mmol) in methanol (150 mL) and concentrated sulfuric acid (3 mL) was heated at reflux for 1.5 hours and then cooled, concentrated under reduced pressure and partitioned between ethyl acetate (100 mL) and water (100 mL). The aqueous layer was further extracted with ethyl acetate (2×50 mL) and the combined organic layers washed with brine (50 mL), dried over anhydrous magnesium sulfate, filtered and evaporated to dryness. After purification by column chromatography on silica gel with 1:1 ethyl acetate as eluent the title compound was obtained as a colourless oil; MS (ES+ve): [M-OH]⁺ at m/z 205 (C₁₃H₁₈O₃ requires [M-OH]⁺ at m/z 205); [α]_{D}^{30° C.}−30.7° (c=1.0, MeOH).

### Preparation 30

### (S)-(+)-3-(4-Hydroxymethylphenyl)pentanoic acid methyl ester (P30)

The title compound was prepared as a colourless oil from (S)-3-(4-hydroxymethylphenyl)pentanoic acid (P28) following the method of Preparation 29; MS (ES+ve): [M-OH]⁺ at m/z 205 (C₁₃H₁₈O₃ requires [M-OH]⁺ at m/z 205); [α]_{D}^{30° C.}+31.4° (c=1.0, MeOH)

### Preparation 31

### (R)-3-(4-Methanesulfonyloxymethylphenyl)pentanoic acid methyl ester (P31)

The title compound was prepared from (R)-(−)-3-(4-hydroxymethylphenyl)pentanoic acid methyl ester (P29) following the method of Preparation 10; MS (ES+ve): [M-OMs]⁺ at m/z 205 (C₁₄H₂₀O₅S requires [M-OMs]⁺ at m/z 205).

### Preparation 32

### (R)-3-(4-{6-Oxo-5-[4-(3-o-tolylureido)phenyl]-6H-pyridazin-1-ylmethyl}phenyl)-pentanoic acid methyl ester (P32)

To 1-[4-(3-oxo-2,3-dihydropyridazin-4-yl)phenyl]-3-o-tolylurea (prepared from 4-(4-aminophenyl)-2H-pyridazin-3-one [described in EP 0138344] by the general method of Preparation 17) (341 mgs, 60% purity, 0.66 mmol) in N,N-dimethylformamide (6 mL) was added cesium carbonate (896 mg, 2.75 mmol) and (R)-3-(4-methanesulfonyloxymethylphenyl)pentanoic acid methyl ester (P31, 330 mg, 1.1 mmol) and the solution stirred for 16 hours at ambient temperature following the method of Preparation 18. Ethyl acetate was added (50 mL), washed with water (2×50 mL) and the organic layer concentrated in vacuo. The compound was purified by silica chromatography with a linear gradient of 10-100% ethyl acetate in hexane. The appropriate fractions were combined to yield the title compound after evaporation to dryness; LC/MS (ES+ve): [M+H]⁺ at m/z 525 (C₃₁H₃₂N₄O₄ requires [M+H]⁺ at m/z 525)

### Preparation 33

### 5-Chloro-4-(3-methoxyphenyl)-2H-pyridazin-3-one (P33)

A solution of 3-methoxyphenylmagnesium bromide in tetrahydrofuran (1M, 100 mL, 100 mmol) was slowly added to a stirred solution of 4,5-dichloro-2H-pyridazin-3-one (6.6 g, 40 mmol) in a mixture of tetrahydrofuran (30 mL) and diethyl ether (100 mL) cooled to 15° C. The mixture was stirred at room temperature for 30 minutes and then cooled in an ice bath. Saturated aqueous ammonium chloride (70 mL) was added slowly. The mixture was diluted with water and the solid collected by filtration. The solid was washed successively with dilute hydrochloric acid, water and diethyl ether, then dried under vacuum. The combined filtrates were extracted with diethyl ether, washed with water, brine, dried over anhydrous magnesium sulfate, filtered and evaporated to dryness. This residue was crystallised from ethyl acetate to give a further batch of product as a white solid; MS (ES+ve): [M+H]⁺ at m/z 237/239 (C₁₁HgClN₂O₂ requires [M+H]⁺ at m/z 237/239).

### Preparation 34

### 4-(3-Methoxyphenyl)-2H-pyridazin-one (P34)

5-Chloro-4-(3-methoxyphenyl)-2H-pyridazin-3-one (P33, 8.22 g, 34.7 mmol) was dissolved in a solution of sodium hydroxide (3.48 g, 87 mmol) in water (100 mL) and N,N-dimethylformamide (12 mL). 10% Palladium on carbon (0.3 g) was added and the mixture shaken under hydrogen (50 psi) at room temperature for 3 hours. 2M Sodium hydroxide was added to dissolve a precipitate and the filtered solution acidified with concentrated hydrochloric acid. The resulting white solid was collected by filtration, washed with water and dried under vacuum to give the title compound; MS (ES+ve): [M+H]⁺ at m/z 203 (C₁₁H₁₀N₂O₂ requires [M+H]⁺ at m/z 203).

### Preparation 35

### N-[2-Methoxy-4-(3-oxo-2,3-dihydropyridazin-4-yl)phenyl]acetamide (P35)

4-(3-Methoxyphenyl)-2H-pyridazin-3-one (P34, 4.0 g, 19.8 mmol) was added portionwise to a stirred mixture of concentrated nitric acid (16 mL) and concentrated sulfuric acid (1.6 mL) at 15° C. The mixture was stirred at room temperature for 4 hours then added to a rapidly stirred ice-water mixture (300 mL). The pale yellow solid was collected by filtration, washed with water and dried under vacuum to give a mixture of 4-(3-methoxy-2-nitrophenyl)-2H-pyridazin-3-one, 4-(3-methoxy-4-nitrophenyl)-2H-pyridazin-3-one, and 4-(5-methoxy-2-nitrophenyl)-2H-pyridazin-3-one; MS (ES+ve): [M+H]⁺ at m/z 248 (C₁₁H₉N₃O₄ requires [M+H]⁺ at m/z 248).

The mixture of nitro isomers obtained above (4.76 g) was dissolved in solution of sodium hydroxide (1.64 g, 41 mmol) in water (120 mL) and N,N-dimethylformamide (14 mL). 10% Palladium on carbon (0.2 g) was added and the mixture shaken under hydrogen (15 psi) at room temperature for 16 hours. 2M Sodium hydroxide (6 mL) was added to dissolve a precipitate, a further portion of 10% palladium on carbon (0.2 g) was added, and hydrogenation was continued for a further 6 hours. The filtered solution was acidified to pH 2.0 by addition of concentrated hydrochloric acid. The solution was evaporated to dryness and the residue containing a mixture of 4-(2-amino-3-methoxyphenyl)-2H-pyridazin-3-one, 4-(4-amino-3-methoxyphenyl)-2H-pyridazin-3-one and 4-(2-amino-5-methoxyphenyl)-2H-pyridazin-3-one was dried under vacuum at 40° C. overnight.

The above mixture was dissolved in water (350 mL), sodium acetate trihydrate (30 g) was added and the mixture cooled in an ice bath. Acetic anhydride (25 mL) was added. After 10 minutes the ice bath was removed and stirring was continued for a further 30 minutes. The mixture was evaporated to dryness and the residue was extracted with a dichloromethane/methanol mixture (9:1, 250 mL). The extract was partially purified by chromatography (silica gel, 5-10% methanol in dichloromethane) to give, in order of elution: N-[2-methoxy-4-(3-oxo-2,3-dihydropyridazin-4-yl)phenyl]acetamide, N-[4-methoxy-2-(3-oxo-2,3-dihydropyridazin-4-yl)phenyl]acetamide and N-[2-methoxy-6-(3-oxo-2,3-dihydropyridazin-4-yl)phenyl]acetamide. Crystallisation of the earliest eluting isomer from ethyl acetate gave pure N-[2-methoxy-4-(3-oxo-2,3-dihydropyridazin-4-yl)phenyl]acetamide; MS (ES+ve): [M+H]⁺ at m/z 260 (C₁₃H₁₃N₃O₃ requires [M+H]⁺ at m/z 260).

### Preparation 36

### 4-(4-Amino-3-methoxyphenyl)-2H-pyridazin-3-one (P36)

A mixture of N-[2-methoxy-4-(3-oxo-2,3-dihydropyridazin-4-yl)phenyl]acetamide (P35, 0.42 g) and 6M hydrochloric acid (20 mL) was heated under reflux for 30 minutes. After cooling and concentrating, the residue was dissolved in dilute sodium hydroxide. Dilute hydrochloric acid was added to pH 5-6 with ice bath cooling. The resulting solid was collected by filtration, washed with cold water and dried under vacuum to give the title compound; MS (ES+ve): [M+H]⁺ at m/z 218 (C₁₁H₁₁N₃O₂ requires [M+H]⁺ at m/z 218).

### Preparation 37

### 2-Methoxy-3-(3-methoxy nitrophenyl)pyrazine (P37)

2,2,6,6-Tetramethylpiperidine (0.71 mL, 4.21 mmol) was added to a solution of n-butyllithium (1.6M, 2.6 mL, 4.16 mmol) in tetrahydrofuran (10 mL) at −30° C. The mixture was allowed to warm up to 0° C. and stirred at that temperature for 15 minutes. The solution was then cooled to −70° C., a solution of 2-methoxypyrazine (200 mg, 1.80 mmol) in tetrahydrofuran (5 mL) was added and then the mixture stirred at that temperature for 30 minutes. A solution of zinc chloride (500 mg, 3.67 mmol) in tetrahydrofuran (5 mL) was subsequently added at −70° C. and the mixture was then allowed to warm slowly to room temperature. A solution containing tetrakis(triphenylphosphine)palladium (0) (83 mg, 0.07 mmol) and 4-bromo-2-methoxy-1-nitrobenzene (459 mg, 1.98 mmol) in tetrahydrofuran (5 mL) was added to the organozinc derivative and the mixture heated at 65° C. for 2 hours. The reaction mixture was then hydrolysed with a solution containing ethylenediaminetetraacetic acid (1.1 g, 3.7 mmol) in water (10 mL) which had been made slightly basic with a saturated aqueous solution of potassium carbonate. The aqueous phase was extracted with dichloromethane (3×100 mL) and the combined extracts dried over magnesium sulfate and concentrated in vacuo. The product was purified by silica gel chromatography eluting with 0 to 80% ethyl acetate in hexane to yield the title compound as a solid; MS (APCI+ve): [M+H]⁺ at m/z 262 (C₁₂H₁₁N₃O₄ requires [M+H]⁺ at m/z 262).

### Preparation 38

### 3-(3-Methoxy-4-nitrophenyl)-1H-pyrazin-2-one (P38)

Thionyl chloride (2 mL) was added to a solution of 2-methoxy-3-(3-methoxy-4-nitrophenyl)pyrazine (P37, 340 mg, 1.30 mmol) in ethanol (10 mL). The reaction mixture was heated to reflux for 24 hours and then concentrated in vacuo to yield the title compound as a solid; MS (APCI+ve): [M+H]⁺ at m/z 248 (C₁₁H₉N₃O₄ requires [M+H]⁺ at m/z 248).

### Preparation 39

### N-Acetyl-2-(4-nitrophenyl)acetamide (P39)

2-(4-Nitrophenyl)acetamide (7.25 g, 40.3 mmol) was stirred in acetic anhydride (30 mL) as boron trifluoride-acetic acid complex (1.5 mL, 10.8 mmol) was added. The mixture was stirred for 4 days, treated with a further portion of boron trifluoride-acetic acid complex (3.0 mL, 21.6 mmol), and stirred for a further day. It was then diluted with a solution of sodium acetate (50 g) in water (250 mL), warmed to 100° C. for 20 minutes, and cooled to ambient temperature. The solid was filtered off and washed with water, yielding the title compound as a powder; LC/MS (ES-ve): [M−H] at m/z 221 (C₁₀H₁₀N₂O₄ requires [M−H]⁻ at m/z 221).

### Preparation 40

### 2,6-Dimethyl-5-(4-nitrophenyl)-4-(1H)-pyrimidinone (P40)

N-Acetyl-2-(4-nitrophenyl)acetamide (P39, 3.12 g, 14.1 mmol) and boron trifluoride-acetic acid complex (7.5 mL, 54.0 mmol) were stirred in acetic anhydride (100 mL) at 60° C. for 20 hours, cooled, and evaporated to dryness in vacuo. Acetic acid (100 mL) and ammonium acetate (8 g) were added, and the mixture was stirred at reflux for 1 hour before evaporating again to dryness in vacuo. The residue was taken up in water (100 mL) and ethyl acetate (50 mL), and neutralised with saturated aqueous sodium hydrogen carbonate. The solid was filtered off, washed with ethyl acetate and then water, and dried in vacuo to give the title compound as a powder; LC/MS (ES+ve): [M+H]⁺ at m/z 246 (C₁₂H₁₁N₃O₃ requires [M+H]⁺ at m/z 246).

### Preparation 41

### Methyl (3R)-3-(4-{[2,4-dimethyl-5-(4-nitrophenyl)-6-oxo-1(6H)-pyrimidinyl]methyl}phenyl)butanoate (P41)

2,6-Dimethyl-5-(4-nitrophenyl)-4(1H)-pyrimidinone (P40, 0.25 g, 1.02 mmol), (R)-3-(4-Methanesulfonyloxymethylphenyl)butyric acid methyl ester (P10, 0.337 g, 1.18 mmol) and cesium carbonate (0.67 g, 2.06 mmol) were stirred in dry N,N-dimethylformamide (10 mL) for 16 hours, diluted with ethyl acetate (50 mL), washed with water (2×) and brine, dried over anhydrous magnesium sulfate and evaporated in vacuo. Purification by flash chromatography on silica gel, eluting with 20-100% ethyl acetate in hexane, gave initially the presumed O-alkylated material, followed by the title compound as a solid after evaporation to dryness; LC/MS (ES+ve): [M+H]⁺ at m/z 436 (C₂₄H₂₅N₃O₅ requires [M+H]⁺ at m/z 436).

### Preparation 42

### Methyl (3R)-3-(4-{[5-(4-aminophenyl)-2,4-dimethyl-6-oxo-1(6H)-pyrimidinyl]-methyl}phenyl)butanoate (P42)

Methyl (3R)-3-(4-{[2,4-dimethyl-5-(4-nitrophenyl)-6-oxo-1(6H)-pyrimidinyl]methyl}phenyl)butanoate (P41, 0.266 g, 0.611 mmol) and tin (II) chloride dihydrate (0.69 g, 3.06 mmol) were stirred at reflux in a mixture of ethanol (10 mL) and ethyl acetate (10 mL) for 2 hours, cooled, and treated with excess solid sodium hydrogen carbonate. The mixture was filtered through kieselguhr, washed with saturated sodium hydrogen carbonate solution, dried over anhydrous magnesium sulfate, and evaporated to dryness in vacuo, giving the title compound as a gum; LC/MS (ES+ve): [M+H]⁺ at m/z 406 (C₂₄H₂₇N₃O₃ requires [M+H]⁺ at m/z 406).

### Preparation 43

### Methyl (3R)-3-(4-{[2,4-dimethyl-5-[4-({[(2-methylphenyl)amino]carbonyl}amino)-phenyl]-6-oxo-1(6H)-pyrimidinyl]methyl}phenyl)butanoate (P43)

Methyl (3R)-3-(4-{[5-(4-aminophenyl)-2,4-dimethyl-6-oxo-1(6H)-pyrimidinyl]methyl}phenyl) butanoate (P42, 0.225 g, 0.555 mmol) and o-tolyl isocyanate (0.083 mL, 0.670 mmol) were stirred in dry dichloromethane (10 mL) for 16 hours. The reaction mixture was applied directly to a flash silica column, and eluted with 40-100% ethyl acetate in hexane, giving the title compound still contaminated with starting material. The reaction was repeated, stirring over a period of 4 days. Chromatographic purification as above then gave the pure title compound as a gum; LC/MS (ES+ve): [M+H]⁺ at m/z 539 (C₃₂H₃₄N₄O₄ requires [M+H]⁺ at m/z 539).

### Preparation 44

### [3-Methoxy-4-nitrophenyl]acetonitrile (P44)

2-Nitroanisole (8.0 mL, 65.5 mmol) and [(4-chlorophenyl)oxy]acetonitrile (12.0 g, 71.6 mmol) were dissolved in dry N,N-dimethylformamide (50 mL) and added dropwise to a stirred solution/suspension of potassium t-butoxide (16.1 g, 143.7 mmol) in dry N,N-dimethylformamide (100 mL) at −20° C. The mixture was stirred at −20° C. for 30 min, poured into ice/2M hydrochloric acid, and stirred for 1 hour. The resulting semi-solid was filtered off, washed with water, dissolved in ethyl acetate, dried over anhydrous magnesium sulfate, and evaporated in vacuo to give a black oil (17.14 g). Purification by flash chromatography on silica gel, eluting with 0-60% ethyl acetate in hexane gave, as the faster eluting isomer, the title compound as a solid; LC/MS (ES-ve): [M−H]⁻ at m/z 191 (C₉H₈N₂O₃ requires [M−H]⁻ at m/z 191).

### Preparation 45

### [3-Methoxy 4-nitrophenyl]acetic acid (P45)

[3-Methoxy-4-nitrophenyl]acetonitrile (P44, 2.43 g, 12.7 mmol) was stirred at reflux in concentrated hydrochloric acid (50 mL) for 1 hour. The mixture was cooled, evaporated to dryness, and triturated with water. The solid was filtered off and dried, giving the title compound as a solid; LC/MS (ES+ve): [M+H]⁺ at m/z 212 (C₉H₉NO₅ requires [M+H]⁺ at m/z 212).

### Preparation 46

### 2-[3-Methoxy-4-nitrophenyl]acetamide (P46)

[3-Methoxy-4-nitrophenyl]acetic acid (P45, 1.70 g, 8.1 mmol) was stirred at reflux in thionyl chloride (10 mL) for 1 hour, and then evaporated to dryness. The residue was dissolved in dry tetrahydrofuran (20 mL), and added slowly to aqueous ammonia (d 0.88, mL) with efficient stirring. After standing for 3 days, the mixture was diluted with water (100 mL) and extracted with ethyl acetate. The extract was dried over anhydrous magnesium sulfate and evaporated to give the title compound as a solid; LC/MS (ES-ve): [M−H]⁻ at m/z 209 (C₉H₁₀N₂O₄ requires [M−H]⁻ at m/z 209).

### Preparation 47

### 2,6-Dimethyl-5-[3-methoxy-4-nitrophenyl]-4(1H)-pyrimidinone (P47)

2-[3-Methoxy-4-nitrophenyl]acetamide (P46, 1.23 g, 5.86 mmol) and boron trifluoride-acetic acid complex (3.3 mL, 23.7 mmol) were stirred in acetic anhydride (30 mL) at 60° C. for 16 hours, cooled, and evaporated to dryness in vacuo. Acetic acid (30 mL) and ammonium acetate (4 g) were added, and the mixture was stirred at reflux for 1 hour before evaporating again to dryness in vacuo. The residue was taken up in water (100 mL) and ethyl acetate (50 mL), and neutralised with saturated sodium hydrogen carbonate solution. The solid was filtered off, washed with ethyl acetate and then water, and dried in vacuo to give the title compound as a powder; LC/MS (ES+ve): [M+H]⁺ at m/z 276 (C₁₃H₁₃N₃O₄ requires [M+H]⁺ at m/z 276).

### Preparation 48

### Methyl (3R)-3-(4-{[2,4-dimethyl-5-[3-methoxy-4-nitrophenyl]-6-oxo-1(6H-pyrimidinyl]methyl}phenyl)butanoate (P48)

2,6-Dimethyl-5-[3-methoxy-4-nitrophenyl]-4(1H)-pyrimidinone (P47, 0.27 g, 0.98 mmol), methyl (3R)-3-(4-{[(methylsulfonyl)oxy]methyl}phenyl)butanoate (P10, 0.337 g, 1.18 mmol) and cesium carbonate (0.67 g, 2.06 mmol) were stirred in dry N,N-dimethylformamide (10 mL) for 16 hours, diluted with ethyl acetate (50 mL), washed with water (x2) and brine, dried over anhydrous magnesium sulfate and evaporated in vacuo. Purification by flash chromatography on silica gel, eluting with 20-100% ethyl acetate in hexane, gave initially the presumed O-alkylated material, followed by the title compound, the latter as a gum; LC/MS (ES+ve): [M+H]⁺ at m/z 466 (C₂₅H₂₇N₃O₆ requires [M+H]⁺ at m/z 466).

### Preparation 49

### Methyl (3R)-3-(4-{[5-[4-amino-3-methoxyphenyl]-2,4-dimethyl-6-oxo-1(6H)-pyrimidinyl]methyl}phenyl)butanoate (P49)

Methyl (3R)-3-(4-{([2,4-dimethyl-5-[3-methoxy-4-nitrophenyl]-6-oxo-1(6H-pyrimidinyl]methyl}phenyl)butanoate (P48, 0.246 g, 0.529 mmol) and tin (II) chloride dihydrate (0.60 g, 2.66 mmol) were stirred at reflux in a mixture of ethanol (10 mL) and ethyl acetate (10 mL) for 2 hour, cooled, and treated with excess solid sodium hydrogen carbonate. The mixture was filtered through kieselguhr, washed with saturated sodium hydrogen carbonate solution, dried over anhydrous magnesium sulfate, and evaporated to dryness in vacuo, giving the title compound as a gum; LC/MS (ES+ve): [M+H]⁺ at m/z 436 (C₂₅H₂₉N₃O₄ requires [M+H]⁺ at m/z 436).

### Preparation 50

### Methyl (3R)-3-(4-{[2,4-dimethyl-5-[3-methoxy-4-({[(2-methylphenyl)amino]-carbonyl}amino)phenyl]-6-oxo-1(6″-pyrimidinyl]methyl}phenyl)butanoate (P50)

Methyl (3R)-3-(4-{[5-[4-amino-3-methoxyphenyl]-2,4-dimethyl-6-oxo-1(6H)-pyrimidinyl]-methyl}phenyl)butanoate (P49, 0.184 g, 0.423 mmol) and o-tolyl isocyanate (0.063 mL, 0.509 mmol) were stirred in dry dichloromethane (10 mL) for 16 hours. The reaction mixture was applied directly to a flash silica column, and eluted with 40-100% ethyl acetate in hexane, giving the title compound still contaminated with starting material. The reaction was repeated, stirring over a period of 4 days. Chromatographic purification as above then gave the title compound as a gum; LC/MS (ES+ve): [M+H]⁺ at m/z 569 (C₃₃H₃₆N₄O₅ requires [M+H]⁺ 569)

### EXAMPLES

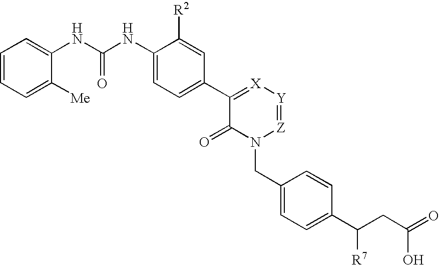

| Com- | | | | | | Calc. | Observed |
|---|---|---|---|---|---|---|---|
| pound | X | Y | Z | R² | R⁷ | Mass M | [M + H]⁺ |
| E1 | N | CH | CH | H | (R)-Me | 496.571 | 497 |
| E2 | N | CH | CH | H | (R)-Et | 510.598 | 511 |
| E3 | CH | N | CH | H | H | 482.544 | 483 |
| E4 | CH | N | CH | H | (R)-Me | 496.571 | 497 |
| E5 | CH | N | CH | H | (R)-Et | 510.598* | 511 |
| E6 | CH | N | CH | H | (S)-Et | 510.598* | 511 |
| E7 | CH | N | CH | MeO | (R)-Me | 526.597 | 527 |
| E8 | CH | N | CH | MeO | (S)-Me | 526.597 | 527 |
| E9 | CH | N | CH | EtO | (R,S)-Me | 540.624 | 541 |
| E10 | CH | CH | N | H | (R)-Me | 496.571 | 497 |
| E11 | CH | CH | N | H | (R)-Et | 510.598* | 511 |
| E12 | CH | CH | N | H | (S)-Et | 510.598 | 511 |
| E13 | CH | CH | N | MeO | (R)-Me | 526.597 | 527 |
| E14 | N | CH | CH | MeO | (R)-Me | 526.597 | 527 |
| E15 | CMe | N | CMe | H | (R,S)-Me | 524.625 | 525 |
| E16 | CMe | N | CMe | H | (R)-Me | 524.625 | 525 |
| E17 | CMe | N | CMe | H | (S)-Me | 524.625 | 525 |
| E18 | CMe | N | CMe | MeO | (R)-Me | 554.651 | 555 |

*Compounds prepared as Na salt: calculated mass of parent acid shown

The above tabulated compounds E1-E18 were prepared using the methodology described below.

### Example 9

### (R,S)-3-(4-{5-[3-Ethoxy-4-(3-o-tolylureido)phenyl]-6-oxo-6H-pyrimidin-1-ylmethyl}phenyl)butyric acid (E9)

A solution of (R,S)-3-(4-{5-[3-ethoxy-4-(3-o-tolylureido)phenyl]-6-oxo-6H-pyrimidin-1-ylmethyl}phenyl)butyric acid ethyl ester (P18, 348 mg, 0.61 mmol) in tetrahydrofuran (16 mL) was treated with 0.5N aqueous lithium hydroxide solution (13 mL). The reaction mixture was stirred for 16 hours and then acidified with 2N hydrochloric acid. The residue was diluted with ethyl acetate (20 mL) and after separation of the organic layer, the aqueous phase was re-extracted with ethyl acetate (2×20 mL). The combined organic layers were dried over magnesium sulfate, filtered and concentrated at reduced pressure to yield the title compound as a colourless solid; ¹H NMR δ (DMSO-d6): 1.18 (3H, d), 1.41 (3H, t), 2.27 (3H, s), 2.46 (2H, m), 3.15 (1H, m), 4.18 (2H, q), 5.15 (2H, s), 6.97 (1H, ap. t), 7.15 (1H, ap. t), 7.18 (1H, d), 7.27 (5H, m), 7.42 (1H, d), 7.71 (1H, d), 8.13 (1H, d), 8.19 (1H, s), 8.48 (1H, s), 8.63 (1H, s), 8.67 (1H, s), 12.05 (1H, br. s); LC/MS (ES+ve) [M+H]⁺ at m/z 541 (C₃₁H₃₂N₄O₅ requires [M+H]⁺ at m/z 541).

The corresponding chiral methoxy substituted compounds E7 and E8 were prepared similarly to Preparations 14-18 starting from the known (3-methoxy-4-nitrophenyl)acetonitrile [PCT Int. Appl. WO 86/01204] except that the hydrolysis of acetonitrile to acetamide was conducted over 16 hours instead of 48 hours.

### Example 4

### (R)-3-(4-{6-Oxo-5-[4-(3-o-tolylureido)phenyl]-6H-pyrimidin-1-ylmethyl}phenyl)butyric acid (E4)

(R)-3-(4-{6-Oxo-5-[4-(3-o-tolylureido)phenyl]-6H-pyrimidin-1-ylmethyl}phenyl)butyric acid methyl ester (P21, 380 mg, 0.75 mmol) in tetrahydrofuran (10 mL) was stirred with 0.5N lithium hydroxide (10 mL) for 3 hours at room temperature. The reaction mixture was acidified to pH 1 with 2N aqueous hydrochloric acid and extracted with ethyl acetate. The organic layer was dried over magnesium sulfate, filtered and concentrated in vacuo to yield the title compound as a solid; MS (ES+ve): [M+H]⁺ at m/z 497 (C₂₉H₂₈N₄O₄ requires [M+H]⁺ at m/z 497); ¹H NMR δ (DMSO-d6): 1.18 (3H, d), 2.24 (3H, s), 2.50 (2H, d), 3.14 (1H, q), 5.13 (2H, s), 6.93 (1H, t), 7.13 (2H, m), 7.25 (2H, d), 7.32 (2H, d), 7.50 (2H, d), 7.65 (2H, d), 7.85 (1H, d), 7.96 (1H, s), 8.12 (1H, s), 8.65 (1H, s), 9.16 (1H, s), 11.92 (1H, s).

Compounds E1 and E2 were prepared from 3-(4-aminophenyl)-1H-pyrazin-2-one (P22) using methods analogous to those disclosed in relevant Preparations and Examples herein. For example E1 is prepared by reaction of P22 with o-tolylisocyanate by the method of Preparation 17, the resulting pyrazinone then alkylated with (R)-3-(4-methanesulfonyloxymethylphenyl)butyric acid methyl ester (P10), and the resulting ester hydrolysed by the method of Example 9.

Compounds E10-E12 are prepared from 4-(4-aminophenyl)-2H-pyridazin-3-one [described in EP 0138344] in an analogous manner to that outlined for E1 above using the appropriate alkylating agents.

### Example 11

### (R)-3-(4-{6-Oxo-5-[4-(3-o-tolylureido)phenyl]-6H-pyridazin-1-ylmethyl}phenyl)-pentanoic acid sodium salt (E11)

To (R)-3-(4-{6-oxo-5-[4-(3-o-tolylureido)phenyl]-6H-pyridazin-1-ylmethyl}phenyl) pentanoic acid methyl ester (P32) in tetrahydrofuran (5 mL) was added 0.5M lithium hydroxide (5 mL) and the solution stirred at ambient temperature for 3 hours. 10% Citric acid solution was added until pH 5 attained and the product extracted into ethyl acetate (2×50 mL) and then washed with water (2×50 mL). The organic layer was concentrated and then purified by chromatography on silica gel with a linear gradient of 0-10% methanol in dichloromethane as eluent. The appropriate fractions were combined and the solution concentrated. Sodium hydroxide (2M, 93 μL, 1 equiv.) was added and the solution concentrated again to yield the title compound as a white solid; LC/MS (ES+ve): [M+H]⁺ at m/z 511 (C₃₀H₃₀N₄O₄ (free acid) requires [M+H]⁺ at m/z 511); ¹H NMR δ (DMSO-d6): 0.67 (3H, t), 1.44 (1H, m), 1.67 (1H, m), 2.16 (1H, dd), 2.22 (1H, d), 2.24 (3H, s), 2.93 (1H, m), 5.24 (1H, d), 5.30 (1H, d), 6.93 (2H, dd), 7.11 (1H, dd), 7.14 (1H, d), 7.15 (2H, d), 7.23 (2H, d), 7.53 (2H, d), 7.59 (2H, d), 7.62 (1H, d), 7.81 (1H, d), 7.92 (1H, d), 9.84 (1H, br. s), 11.12 (1H, br. s).

Other chiral ethyl substituted compounds E2, E5, E6, and E12 were prepared in analogous manner to compound E11.

### Example 13

### (R)-3-(4-{5-[3-Methoxy-4-(3-o-tolylureido)phenyl]-6-oxo-6H-pyridazin-1-ylmethyl}-phenyl)butyric acid (E13)

The title compound is prepared from P36 by firstly reaction with o-tolylisocyanate by the method of Preparation 17 to give 1-[2-methoxy-4-(3-oxo-2,3-dihydropyridazin-4-yl)-phenyl]-3-o-tolylurea, then alkylation with the mesylate P10 by the general method of Preparation 18 to give (R)-3-(4-{5-[3-methoxy-4-(3-o-tolylureido)phenyl]-6-oxo-6H-pyridazin-1-ylmethyl}phenyl)butyric acid methyl ester.

(R)-3-(4-{5-[3-Methoxy-4-(3-o-tolylureido)phenyl]-6-oxo-6H-pyridazin-1-ylmethyl}phenyl)butyric acid methyl ester (84 mg, 0.155 mmol) in tetrahydrofuran (5 mL) was then treated with 0.5M lithium hydroxide (7 mL) and stirred at room temperature for 6.5 hours, then product isolated by the method of Example 9; MS (ES+ve): [M+H]⁺ at m/z 527 (C₃₀H₃₀N₄O₅ requires [M+H]⁺ at m/z 527); ¹H NMR δ (DMSO-d6): 1.18 (3H, d), 2.26 (3H, s), 2.47 (2H, obscured by solvent), 3.11 (1H, m), 3.94 (3H, s), 5.30 (2H, s), 6.96 (1H, t), 7.12-7.28 (7H, m), 7.52 (2H, dd), 7.66 (2H, m), 7.80 (1H, d), 8.00 (2H, d), 8.23 (1H, d), 8.61 (1H, s), 8.83 (1H, s), 12.06 (1H, s).

### Example 14

### (R)-3-(4-{3-[3-methoxy-(3-o-tolylureido)phenyl]-2-oxo-2H-pyrazin-1-ylmethyl}-phenyl)butyric acid (E14)

The title compound was prepared from P38 by analogous procedures to those described herein, i.e. 3-(3-methoxy-4-nitrophenyl)-1H-pyrazin-2-one (P38) is converted to (R)-3-{4-[3-(3-methoxy-4-nitrophenyl)-2-oxo-2H-pyrazin-1-ylmethyl]phenyl}butyric acid methyl ester by the method of Preparation 19; reduced to (R)-3-{4-[3-(4-amino-3-methoxyphenyl)-2-oxo-2H-pyrazin-1-ylmethyl]phenyl}butyric acid methyl ester by the method of Preparation 20. This amine is then converted to the urea (R)-3-(4-{3-[3-methoxy-4-(3-o-tolyl-ureido)phenyl]-2-oxo-2H-pyrazin-1-ylmethyl}phenyl)butyric acid methyl ester by the method of Preparation 21 and finally hydrolysis of the methyl ester by the method of Example 4 affords the title compound.

### Example 16

### (3R)-3-(4-{[2,4-Dimethyl-5-[4-({[(2-methylphenyl)amino]carbonyl}amino)phenyl]-6-oxo-1(6H)-pyrimidinyl]methyl}phenyl)butanoic acid (E16)

Methyl (3R)-3-(4-{[2,4-dimethyl-5-[4-({[(2-methylphenyl)amino]carbonyl}amino)phenyl]-6-oxo-1(6H)-pyrimidinyl]methyl}phenyl)butanoate (P43, 0.230 g, 0.427 mmol) was stirred in a mixture of tetrahydrofuran (5 mL) and 0.5M lithium hydroxide (5 mL) for 4 hours. The mixture was diluted with water, washed with ether, acidified with 2M hydrochloric acid and extracted into ethyl acetate. As the extract was being dried over anhydrous magnesium sulfate, precipitation started to occur, so the drying agent was washed well with 20% methanol in dichloromethane. Evaporation of the filtrate gave a white solid, which was taken up in water. After filtration, washing with water, and drying the title compound was obtained as a white solid; LC/MS (ES+ve): [M+H]⁺ at m/z 525 (C₃₁H₃₂N₄O₄ requires [M+H]⁺ at m/z 525).

### Example 18

### (3R)-3-(4-{[2,4-Dimethyl-5-[3-methoxy-4-({[(2-methylphenyl)amino]carbonyl}-amino)phenyl]-6-oxo-1(6H)-pyrimidinyl]methyl}phenyl)butanoic acid (E18)

Methyl (3R)-3-(4-{[2,4-dimethyl-5-[3-methoxy-4-({[(2-methylphenyl)amino]carbonyl}amino)phenyl]-6-oxo-1(6H)-pyrimidinyl]methyl}phenyl)butanoate (P50, 0.200 g, 0.352 mmol) was stirred in a mixture of tetrahydrofuran (5 mL) and 0.5M lithium hydroxide (5 mL) for 2.5 hours. The mixture was diluted with water, washed with ether, acidified with 2M hydrochloric acid and extracted into ethyl acetate. The extract was dried over anhydrous magnesium sulfate and evaporated in vacuo to give an off-white semi-solid. This was purified by preparative HPLC, giving the title compound as a white solid; LC/MS (ES+ve): [M+H]⁺ at m/z 555 (C₃₂H₃₄N₄O₅ requires [M+H]⁺ at m/z 555).

1. A compound of formula (I) or a pharmaceutically acceptable derivative thereof: 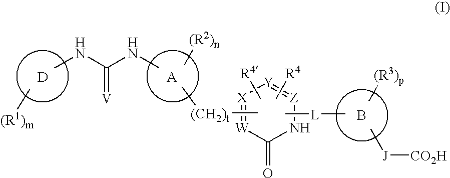
wherein
A, B and D are independently aryl or heteroaryl;
R¹, R² and R³ are independently C₁₋₆alkyl, halogen, C₁₋₆alkoxy, hydroxy, cyano, CF₃, OCF₃, nitro, C₁₋₆alkylthio, amino, mono- or di-C₁₋₆alkylamino, carboxy, C₁₋₆alkanoyl, amido, mono or di-C₁₋₆alkyl amido, —NHCOR⁹ or —NHSO₂R⁹ {in which R⁹ is C₁₋₆alkyl, C₃₋₇cycloalkyl or phenyl (optionally substituted by up to three groups selected from C₁₋₆alkyl, halogen, C₁₋₆alkoxy, cyano, phenyl and CF₃)} or is a group -E-(CH₂)₁₋₆NR^{x}R^{y} (in which E is a single bond or —OCH₂— and R^{x} and R^{y} are independently hydrogen, C₁₋₆alkyl or combine together to form a 5-7 membered heterocyclic ring);
R⁴ and R^{4′} are independently hydrogen, C₁₋₆alkyl, halogen or C₁₋₆alkoxy;
V is O, S, NH, N—C₁₋₆alkyl, NNO₂ or NCN;
W, X, Y and Z are independently C, CH or N, subject to the proviso that at least one of X, Y and Z is N;
L is —(CH₂)_{q}— or —(CH₂)_{q}′O— where q is 0, 1, 2 or 3 and q′ is 2 or 3;
J is
(i) a group —CR⁵═CR⁶— where R⁵ and R⁶ are independently hydrogen or C₁₋₆alkyl;
(ii) a group —CHR⁷—CHR⁸— where R⁷ and R⁸ are independently hydrogen, C₁₋₆alkyl, C₃₋₇cycloalkyl, aryl, heteroaryl, a group —NHCOR⁹ or —NHSO₂R⁹ in which R⁹ is as defined above or a group —(CH₂)₁₋₆NR^{x}R^{y} in which R^{x} and R^{y} are as defined above;
(iii) a single bond;
(iv) —CHR⁶— where R⁶ is as defined above; or
(v) a group —O—CHR¹⁰—, —NR¹¹—CHR¹⁰— or —CR¹²R¹³—CHR¹⁰— where R¹⁰ and R¹¹ are independently hydrogen or C₁₋₆alkyl and R¹² and R¹³ are independently C₁₋₆alkyl or R¹² and R¹³ combine together to form a C₃₋₇cycloalkyl or a 5-7 membered heterocyclic ring;
m, n and p are independently 0, 1, 2 or 3; and
t is 0, 1 or 2.

2. The compound according to claim 1, wherein the compound is of formula (I′) or a pharmaceutically acceptable derivative thereof: 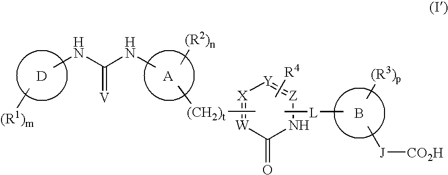
in which R¹-R⁴, m, n, p, t, A, B, D, L, J, V, W, X, Y and Z are as defined in formula (I).

3. The compound according to claim 1, wherein A is phenyl or pyridyl.

4. The compound according to claim 1, wherein B is phenyl.

5. The compound according to claim 1, wherein D is phenyl or pyridyl.

6. The compound according to claim 1, wherein the compound is of formula (Ia) or a pharmaceutically acceptable derivative thereof: 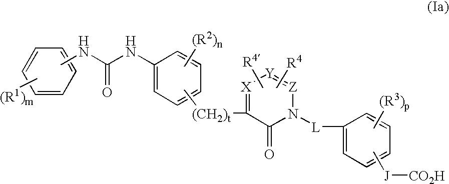
in which:
R¹-R⁴, R^{4′}, L, J, X, Y, Z, m, n, p and t are as defined in formula (I).

7. The compound according to claim 6, wherein the compound is of formula (Ia′) or a pharmaceutically acceptable derivative thereof: 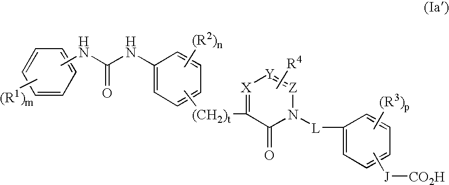
in which:
R¹-R⁴, L, J, X, Y, Z, m, n, p and t are as defined in formula (I).

8. The compound according to claim 1 in which R¹, R² and R³ are, independently, selected from the group consisting of C₁₋₆alkyl, halogen, C₁₋₆alkoxy, cyano, and CF₃.

9. The compound according to claim 1 in which J is selected from the group consisting of —CH═CH—, —(CH₂)₂— and —CHR⁷—CH₂— in which R⁷ is C₁₋₆alkyl.

10. The compound according to claim 1 in which L is —(CH₂)_{q}— where q is 0, 1, 2 or 3.

11. The compound according to claim 1 which is selected from the group consisting of E1-E18 or a pharmaceutically acceptable derivative thereof

12. A process for the preparation of the compound of formula (I) or a pharmaceutically acceptable derivative thereof which comprises hydrolysis of a carboxylic acid ester derivative of formula (II). 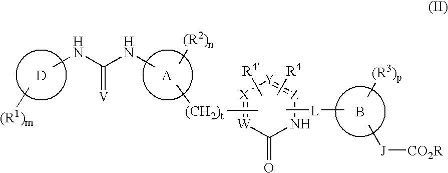
in which R¹-R⁴, R^{4′}, m, n, p, t, A, B, D, L, J, V, W, X, Y and Z are as defined in formula (I) and R is a group capable of forming a carboxylic acid ester and optionally thereafter forming a pharmaceutically acceptable derivative thereof.

13. The compound according to claim 1 for use in therapy.

14. A pharmaceutical composition which comprises a therapeutically effective amount of the compound according to claim 1 in admixture with a pharmaceutically acceptable carrier or diluent.

15. A pharmaceutical composition comprising the compound according to claim 1 together with another therapeutically active agent.

16. A use of the compound according to claim 1 in the manufacture of a medicament for the treatment or prevention of conditions in which an inhibitor of α₄ integrin mediated cell adhesion is beneficial.

17. A method for the treatment or prevention of conditions in which an inhibitor of α₄ integrin mediated cell adhesion is beneficial which comprises administering to a patient in need thereof a safe and effective amount of the compound according to claim 14.

18. The method according to claim 17, wherein said condition is selected from the group consisting of rheumatoid arthritis (RA); asthma; allergic conditions such as rhinitis; adult respiratory distress syndrome; AIDS-dementia; Alzheimer's disease; cardiovascular diseases; thrombosis or harmful platelet aggregation; reocclusion following thrombolysis; reperfusion injury; skin inflammatory diseases such as psoriasis, eczema, contact dermatitis and atopic dermatitis; diabetes (e.g., insulin-dependent diabetes mellitus, autoimmune diabetes); multiple sclerosis; systemic lupus erythematosus (SLE); inflammatory bowel disease such as ulcerative colitis, Crohn's disease (regional enteritis) and pouchitis (for example, resulting after proctocolectomy and ileoanal anastomosis); diseases associated with leukocyte infiltration to the gastrointestinal tract such as Celiac disease, nontropical Sprue, enteropathy associated with seronegative arthropathies, lymphocytic or collagenous colitis, and eosinophilic gastroenteritis; diseases associated with leukocyte infiltration to other epithelial lined tissues, such as skin, urinary tract, respiratory airway, and joint synovium; pancreatitis; mastitis (mammary gland); hepatitis; cholecystitis; cholangitis or pericholangitis (bile duct and surrounding tissue of the liver); bronchitis; sinusitis; inflammatory diseases of the lung which result in interstitial fibrosis, such as hypersensitivity pneumonitis; collagen disease (in SLE and RA); sarcoidosis; osteoporosis; osteoarthritis; atherosclerosis; neoplastic diseases including metastasis of neoplastic or cancerous growth; wound healing enhancement; certain eye diseases such as retinal detachment, allergic conjunctivitis and autoimmune uveitis; Sjogren's syndrome; rejection (chronic and acute) after organ transplantation; host vs. graft or graft vs. host diseases; intimal hyperplasia; arteriosclerosis (including graft arteriosclerosis after transplantation); reinfarction or restenosis after surgery such as percutaneous transluminal coronary angioplasty (PTCA) and percutaneous transluminal artery recanalization; nephritis; tumor angiogenesis; malignant tumor; multiple myeloma and myeloma-induced bone resorption; sepsis; and central nervous system injury such as stroke, traumatic brain injury and spinal cord injury and Meniere's disease.

19. The method according to claim 17, wherein said condition is asthma, allergic conditions, inflammatory bowel disease, rheumatoid arthritis, atopic dermatitis, multiple sclerosis or rejection after organ transplantation.

20. A compound of formula (II): 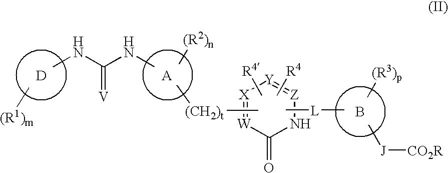
in which R¹-R⁴, R^{4′}, m, n, p, t, A, B, D, L, J, V, W, X, Y and Z are as defined in formula (I) and R is a group capable of forming a carboxylic acid ester.
